(19) United States
(12) Patent Application Publication
(10) Pub. No.: US 2005/0059977 A1
(43) Pub. Date: Mar. 17, 2005

(54) Monitoring circadian activity

(76) Inventor: Jimo Borjigin, Ann Arbor, MI (US)

Correspondence Address: MORGAN LEWIS & BOCKIUS LLP, 1111 PENNSYLVANIA AVENUE NW, WASHINGTON, DC, 20004, US

(21) Appl. No.: 10/812,939

(22) Filed: Mar. 31, 2004

Related U.S. Application Data
(60) Provisional application No. 60/458,440, filed on Mar. 31, 2003.

(51) Int. Cl. A61B 17/16; A61K 49/00

(52) U.S. Cl. 606/80; 424/9.1; 607/88

(57) ABSTRACT

The invention is directed to a new method for long-term measurement of daily serotonin (5-HT) and melatonin contents in the pineal gland. The disclosed method allows visualization of the pineal gland for accurate targeting of the guide cannula which minimizes bleeding, incurs no direct injury to the surrounding brain tissue and cause no interference with the sympathetic innervation from the superior cervical ganglion. The improved method allows effects of pharmacological agents on in vivo pineal gland circulation to be studied reproducibly over time and gene expression profiles correlated with physiological consequences in the same or different individuals. More importantly, the method allows accurate assessment of the endogenous circadian clock function. The method can be used for high throughput screening to identify candidate agents which may accelerate adaptation to new time zones, to alleviate symptoms resulting from jet lag, frequent shift work sleep abnormalities and seasonal affective illnesses.

### CROSS-REFERENCE TO RELATED APPLICATIONS

This application claims priority to U.S. Provisional Application No. 60/458,440, filed Mar. 31, 2003, incorporated herein in its entirety.

### FIELD OF THE INVENTION

This invention relates generally to a surgical method which allows for less invasive surgery and thus improved access to a target tissue of the brain, particularly the pineal. The invention further relates to microdialysis measurement of chemicals, metabolites, agents and tissue infusion and to high throughput screening to identify candidate agents which may accelerate adaptation to new time zones and alleviate symptoms resulting from jet lag, frequent shift work sleep abnormalities and seasonal affective illnesses. The invention also includes methods for modulating the circadian clock of a subject in need thereof.

### BACKGROUND OF THE INVENTION

The pineal gland is a discrete neurosecretory organ of the brain, which functions to synthesize and release the hormone melatonin specifically at night. Melatonin is an important nocturnal signal that informs the body about the time of the day and the season of the year by its regulated amplitude and duration of secretion (Arendt, Chapman and Hall. Melatonin and the Pineal Gland. London (1995)). Melatonin is formed from serotonin (5-hydroxytryptamine, 5-HT) by serotonin N-acetyltransferase (NAT), which produces N-acetylserotonin (NAS), and by hydroxyindole-O-methyltransferase (Borjigin et al., Ann. Rev. Toxic. 39: 53-65 (1999)). Melatonin synthesis is controlled by the rate of NAT mRNA synthesis in the rat pineal gland (Borjigin et al., Nature 378: 783-785 (1995); Borjigin et al., Treatise on Pineal Gland and Melatonin, Science Publishers, Inc., Enfield, 423-430 (2002)), which is regulated by adrenergic innervation from the superior cervical ganglion (Klein et al., Recent Prog. Horm. Res. 52: 307-357 (1997)). Understanding molecular mechanisms of melatonin synthesis and release is important in studying how neuronal signal transduction influence dynamic circadian rhythm generation in the pineal (Borjigin et al., (1999)).

Circadian rhythms are found in virtually every organism and are tightly coupled to environmental lighting conditions. These rhythms dictate daily sleep schedule, daily hormonal fluctuation and even influence susceptibility to disease such as heart attacks, strokes and seizure. One of the best-studied circadian rhythms is the activity of the pineal gland, an organ situated deep within the brain. The pineal gland exhibits dramatic diurnal fluctuations in secretion of the hormone melatonin, which links environmental light information to the body's physiological responses, including clock resetting, seasonal reproduction, and perhaps sleep in all vertebrate animals studied to date. In addition to its physiological role, melatonin is the most reliable marker for the central (circadian) clock activity.

Until about 10 years ago, experimental studies on pineal physiology were conducted with postmortem tissue or sampling of blood. However, studies with postmortem pineals require large numbers of animals and represent only a static picture. Moreover, blood levels of the pineal hormone are not a direct reflection of cellular events that take place in the pineal cells.

Development of microdialysis in the past two decades has greatly increased our understanding of in vivo brain chemistry (Ungerstedt, U., Introduction to Intracerebral Microdialysis. In: Microdialysis in the Neurosciences. Robinson, T. E., Justice, Jr., J. B., Eds. Elsevier, Amsterdam, NY 3-22 (1991)), and had become the preferred method for in vivo sampling (Robinson, T. E. et al., The Feasibility of Repeated Microdialysis for With-In Subjects Design Experiments: Studies on the Mesostriatal Dopamine System. In: Microdialysis in the Neurosciences. Robinson, T. E., Justice, J. B., eds., Elsevier, Amsterdam, NY 189-234 (1991)). Since its adaptation into pineal studies (Azekawa et al., Brain Res. Bull. 26: 413-417 (1991)) and subsequent refinements (Drijfhout et al., J. Neurochem. 61: 936-942 (1993)), it has provided valuable information on the in vivo patterns of pineal secretory activities. Importantly, data obtained by in vivo pineal microdialysis are consistent with and validate those provided by other methods. In addition, in some instances, in vivo pineal microdialysis provides the only measurements of local dynamic pineal secretion of substances such as serotonin (5-HT) (Azekawa et al., Neurosci. Lett. 132: 93-96 (1991)). Similar to other in vivo microdialysis techniques used in neuroscience studies, however, current pineal microdialysis methods permit sampling for no more than two to three days. This sampling duration does not permit constant recovery of substances over the long periods of time (Azekawa et al., 1991) which are required for circadian clock studies.

The major difficulties in pineal microdialysis are due to its location in the brain. The pineal is situated just below the confluence of the superior sagittal sinus and transverse sinus. In order to avoid damaging the blood supply, researchers insert microdialysis probes either diagonally from one side of the brain after opening the skull (Azekawa et al., 1991), or with a transpineal cannula without opening the skull (Drijfhout et al., 1993). Both methods can result in substantial damage of the surrounding brain tissues and suffer from the inability to accurately target the cannula adjacent to the pineal.

Thus, in view of the problems with the known methods discussed above, a new method for implantation of microdialysis probes in the pineal which results in less tissue damage and which provides for long term and continuous monitoring of the chemical output of the pineal is needed.

### SUMMARY OF THE INVENTION

The invention is directed to a method of long term sampling of circadian melatonin output in freely moving individual animals; high resolution mapping of pacemaker function; automated mapping of pacemaker function; and, real-time monitoring of pacemaker activity. The invention comprises a method for implantation of a monitoring device, such as, for example, a microdialysis probe, for monitoring the concentration of a chemical, agent or metabolite in a biological tissue, such as the pineal. In one aspect of the invention, a fluid comprising a chemical, agent or metabolite is guided to a microdialysis probe implanted into, or in close proximity to, the pineal and is discharged therefrom as chemical output after enrichment with a chemical, metabolites or agents from the pineal tissue or pineal tissue fluid. According to the method, other chemicals, agents or metabolites may be added to the chemical output and the concentration in the chemical output is determined.

The invention is also directed to a method for identifying at least one agent which modulates a preselected biological condition controlled by the circadian clock in a subject comprising a) inserting a monitoring device into, or in close proximity to the pineal, causing little or no tissue damage to the non-pineal tissue during the inserting; b) monitoring the chemical output of the pineal and monitoring a preselected biological condition of a first subject; and, c) monitoring the chemical output of the pineal and monitoring the same preselected biological condition as in step b) in a second subject after contacting the second subject with the at least one agent; wherein an alteration in the chemical output of the pineal and in the preselected biological condition in the second subject as compared to the chemical output of the pineal and preselected biological condition in the first subject identifies at least one agent which modulates a preselected biological condition controlled by the circadian clock. The invention is also directed to a composition comprising one or more agents and derivatives thereof identified by the method. In one embodiment, the invention is directed to an agent or derivative thereof identified by the method in purified form. In another embodiment, the invention is directed to a pharmaceutically acceptable composition comprising one or more agents, or derivatives thereof, identified by the method.

The invention is also directed to an improved method of carrying out surgery on the pineal comprising opening the skull of a subject and inserting a monitoring device, the improvement comprising a circular dental disk drill to open the skull, and a hook to lift and/or separate nonpineal tissues away from the pineal to allow visual placement of the monitoring device into, or in close proximity to, the pineal, causing little or no tissue damage to the non-pineal tissue during the implanting.

The invention is further directed to a method for implantation of a microdialysis probe for monitoring of chemicals produced by the pineal, comprising opening the skull and separating nonpineal tissue away from the pineal so as to visually expose the pineal, implanting a microdialysis probe into, or in close proximity to, the pineal, causing little or no tissue damage to the non-pineal tissue during the implanting.

The invention is further directed to a method for monitoring the presence of at least one chemical in the chemical output of the pineal comprising a) opening the skull and visually exposing the pineal; b) inserting a microdialysis probe into, or in close proximity to, the pineal, wherein non-pineal tissue exhibits little or no damage from the inserting; c) contacting the pineal or the subject with the at least one chemical; and, d) monitoring the chemical output of the pineal for presence of the same or different chemical by in vivo microdialysis.

The invention is also directed to a method of modulating a preselected condition controlled by the circadian clock in a subject in need thereof comprising monitoring time of onset of melatonin secretion from said subject prior to presenting a light pulse to said subject; presenting at least one light pulse to said subject, wherein said light pulse is presented during the subject's subjective night phase; and monitoring time of onset of melatonin secretion from said subject after said light pulse; wherein when said melatonin secretion exhibits a shift in the time of onset of secretion after presentation of said light pulse, said preselected condition has been modulated.

### BRIEF DESCRIPTION OF THE DRAWINGS

The foregoing summary, as well as the following detailed description of the invention, will be better understood when read in conjunction with the appended figures. For the purpose of illustrating the invention, there are shown in the figures embodiments which are presently preferred. It should be understood, however, that the invention is not limited to the precise arrangements, examples and instrumentalities shown.

FIGS. 1A-D. Surgical procedure for implantation of the pineal microdialysis probe. 1A. A circular skull incision is made slightly dorsal to the confluence of the sinuses with a dental disk drill. 1B. The underlying dura layer posterior to the confluence of the sinuses (SSS and TS) is carefully removed with a pair of iris forceps under a microscope. Arrowhead indicates the area of cerebellum exposed after the dura matter is peeled away. 1C. The animal's head is repositioned 30 degrees downward, after which the dura connecting the SSS and TS is lifted carefully with a hook fixed to the stereotaxic frame. 1D. The pineal gland with its rich vasculature is clearly visible under the microscope as indicated by the black arrowhead. The white arrowhead points to the major pineal vein, which is avoided when inserting the microdialysis probe.

FIGS. 2A-D. Diurnal profiles of serotonin (5-HT) (horizontal bars), NAS (open squares), and melatonin (filled circles) secretion in 4 classes of operated rats. In all traces, the first night represents 24 hours after the probe placement, and each data point represents the amount of recovery over a 10 minute period. A total of 114 operated rats are divided into 4 classes: class I (panel A), class II (panel B), class III (panel C) and class IV (panel D).

FIG. 3. The effect of repeated ISO treatment on serotonin (5-HT) and melatonin production in a single rat. ISO infusion (1 uM) was given daily between 14:30 and 17:00 from day 7 to day 10.

FIGS. 4A-D. The effect of drug treatment on NAT mRNA induction. Pineal gland of the rat shown in FIG. 2 was infused with ISO (1 uM) on day 11 at 14:30 for 2 hours (panel A). The rat was sacrificed rapidly, the pineal sectioned, and analyzed by in situ hybridization with TPH (lower left panel) and NAT (lower right panel) DIG probes. Rats infused with artificial CSF only (panel B), forskolin (0.1 mg/ml, panel C) and dibutyryl cAMP (10 mM, panel D) were sacrificed at the end of the drug treatments, the pineals processed for in situ hybridization as in panel A. Upper panels in A-D represent the diurnal melatonin (solid black circles) secretion patterns before and after the drug treatment in single animals. The black bars on top of the upper panels in A-D indicate the dark periods (1 AM-11 AM). The stars (*) of the lower panels in A-D indicate the insertion sites of microdialysis probes and the arrowheads represent the probe membranes.

FIG. 5. A free-running rhythm of pineal melatonin secretion in constant darkness determined by on-line pineal microdialysis. SD rats were first entrained by LD12:12 (light intensity at cage level is 300 lux during the light period). The last 3 days of entrainment are shown (D1-D3), followed by constant darkness (D4-D7). The darker shade represents the dark period for the first three days of the experiment.

FIGS. 6A-B. Effect of photoperiod on melatonin secretion upon of the LD transition. PVG rats #1565 and #1928 were entrained in LD14: 10 (for #1565) and LD12:12 (for #1928), respectively. The night period for the initial LD schedules are depicted by the light and intermediate grays. On day 0, the LD cycles were advanced by 6 (#1565) and 8 (#1928) hours by lengthening the dark periods, which is marked by the dark and intermediate grays. Melatonin secretion on the day of the shift (day 0) is marked by the white tracing.

FIGS. 7A-C. Phase angle of melatonin secretion in 2 individual SD rats. Melatonin secretion rhythms are shown for individual rats on consecutive days under entrained conditions. (A and B). The dark period is represented by the gray box. In C, the secretion patterns of the two rats are compared, demonstrating a one hour difference in MT-on.

FIGS. 8A-C. Phase angle of melatonin secretion in 4 strains of rats over a number of circadian cycles. Consecutive day profiles of individual SD (A) and PVG (B) rats are superimposed. Each color represents profiles one individual rat. Daily MT-on and MT-off in a single animal is extremely reproducible; SD rats show higher variation in MT-on compared to PVG rats. C. Superimposition of circadian profiles of multiple rat strains. Each color represents aggregate data from one strain, demonstrating strain dependent clustering of MT-on and MT-off. See text for details.

FIGS. 9A-B. Comparison of rates of melatonin phase shifts during re-entrainment in inbred rats. A. Melatonin secretion in a representative PVG (top) and F344 (bottom) rat was measured before (days −2 to −1) and after (days 0 and up) an 8 hr-advance shift of LD (12:12) cycle. Light and intermediate shaded areas represent the dark periods before the shift, while the dark and intermediate shades represents the dark period after the shift. This illustrate dramatic strain difference in adaption to new LID cycles. B. Aggregate data for reintrainment: Effect of strain. The same shift was presented to 4-6 rats from 4 different strains (LEW-lewis rats in purple; WKY-wistar Kyoto rats in yellow, F344-fisher rats in green, and PVG rats in red). The dark-filled circles represent the MT-on at each cycle, while the white-filled circles the MT-off for each rat at each cycle. The gray areas represent the dark periods. There is strong clustering by strain.

FIG. 10. Melatonin secretion from rat #2038 after LD reversal. Melatonin profiles are shown for 4 days before the shift (D-4 to D-1) and 10 consecutive days following the day and night switch (D 0 to D 9). On day of the shift (DO), there was no nocturnal melatonin peak. The MT-offs for before and after the shift are marked with white-filled circles. Note that the MT-off does not establish the correct phase relationship until day 7, although MT-on is established at day 1.

FIGS. 11A-B. Temporal profile of melatonin rhythms during re-entrainment following an 8 hr-advance shift of the LD cycle. A. Melatonin profiles for individual rats exposed to LD advancement. Clockwise (#1553) and counterclockwise (#1897) shift behavior of melatonin in two SD rats. Following the LD shift on day 0, melatonin of rat #1897 advanced for 20 min, while for rat #1553 melatonin remained unchanged. On day 1, both rats shifted in a clockwise direction, although the #1553 shifted 40 min from the previous steady state position, the #1897 merely returned to where it started from on day 0. On day 2-4, melatonin was undetectable for rat #1553, while for #1897, a steady shift of more than 1 hour per day was observed. On day 5, melatonin suddenly jumped back to the new dark period, which then continued to expand in a clockwise direction. B. Aggregate melatonin profiles for 13 SD rats exposed to LD advancement. The MT-on and MT-off profiles are shown for each rat at each day before and after LD advancement. This experiment demonstrates large heterogeneity in compensation to LD advancement.

### DETAILED DESCRIPTION

Although any methods and materials similar or equivalent to those described herein can be used in the practice or testing of the present invention, the preferred methods and materials are described.

Definitions

Unless defined otherwise, all technical and scientific terms used herein have the same meaning as commonly understood by one of ordinary skill in the art to which this invention belongs.

As used herein, the term “agent” refers to any compound which is pharmacologically active and/or modulates a biological condition in a subject. The terms “chemical,” “metabolite” and “agent” are used interchangeably except where specifically indicated. Where a difference is intended, such will be made clear.

As used herein, the phrase “biological condition” refers to the biological mode, situation, state, status or environment. For example, a biological condition may be the expression of biological molecules of interest such as serotonin or melatonin. Another biological condition may be the state of a particular tissue such as blood levels of hormones. Cortisol levels in blood, for example, may be monitored. Other biological conditions which may be monitored comprise subject behavior, such as sleep patterns and activity patterns, for example.

As used herein, the phrase “biological molecule of interest” refers to any molecule made in or produced by the cell. A biological molecule of interest may also refer to any molecule made by a cell which affects itself and/or a different cell.

As used herein, the phrase “chemical output” refers to chemicals taken up by the dialysis fluid which has contacted the pineal. Chemical output refers to one or more chemicals present in the dialysate. Chemical output of any chemical of the pineal may be measured by any technique disclosed herein or known in the art.

As used herein, the term “circadian clock” refers to the control of all endogenous circadian rhythms. The circadian clock is composed of three parts: light-input pathways, a clock, and effector pathways. Light signals are conveyed by the retina to the suprachiasmatic nuclei (SCN), and the pineal gland produces melatonin (N-acetyl-5-methoxytryptamine), which is regulated by the SCN. Information regarding light is conveyed from the retina to the SCN via the direct retinohypothalamic tract (RHT), as well as indirectly via the lateral geniculate nucleus (LGN). See, for example, U.S. Pat. No. 6,252,073.

As used herein, the term “circadian rhythm(s)” refers to approximately 24-hour oscillations which include the production of biological molecules such as hormones, the regulation of body temperature, and behavior such as wakefulness, alertness, sleep and periods of activity. Circadian rhythms are endogenous, self-sustained oscillations over 24-hour periods found in prokaryotic and eukaryotic organisms. In humans as well as other mammals, the circadian clock, which controls all endogenous circadian rhythms, is located in the SCN of the hypothalamus. One of the most important and reproducible characteristics of a circadian clock is that it can respond to exogenous light/dark signals. See, for example, U.S. Pat. No. 6,525,073.

As used herein, the phrase “derivative” or “derivative thereof” refers to a chemically modified compound, chemical, agent or metabolite wherein the chemical modification takes place at one or more functional groups. The derivative is expected to retain the pharmacologic activity of the compound, chemical, agent or metabolite from which it is derived.

As used herein, the term “melatonergic agents” refers to compounds which have been found to bind human melatonergic receptors expressed in a stable cell line with good affinity. Further, the compounds are agonists as determined by their ability, like melatonin, to block the forskolin-stimulated accumulation of cAMP in certain cells. See, for example, U.S. Pat. No. 6,211,225.

As used herein the term “microdialysis” refers to an investigatory procedure in which a probe is inserted in vivo in tissue such that one side of a semi-permeable membrane will be in contact with tissue and body fluid, while the other side is flushed with a dialysis liquid which takes up substances through the membrane. These substances can then be analyzed in the liquid that flows past (dialysate). See, for example, U.S. Pat. No. 5,735,832.

As used herein, the term “monitoring” refers to analysis, testing or measuring the chemicals in the chemical output of the pineal. “Monitoring” encompasses short term, long term, continuous, periodic measurements and any combination thereof. By “short term” is meant 72 hours or less. By “long term” is meant more than 72 hours and, includes any length of time up to and including two weeks and two moths. “Long term” also includes the amount of time necessary to measure the circadian rhythms of the subject and may include the length of time corresponding to the life span of the subject.

As used herein, the term “pharmaceutically acceptable” means that the agent is compatible with other ingredients of the formulation or composition and not injurious to the patient. Several pharmaceutically acceptable ingredients are known in the art and official publications such as THE UNITED STATES PHARMACOPEIA describe the analytical criteria used to assess the pharmaceutical acceptability of numerous ingredients of interest. The phrase “pharmaceutically acceptable” is used herein to mean that the material so described can be used for treatments in or on animals, including humans, without causing ill effects such as toxicity, for example.

As used herein, the term “subject” broadly refers to any animal that is to be treated with the agents and by the methods disclosed herein. In a preferred embodiment, the term includes humans in need of, or desiring modulation of, a preselected biological condition controlled by the circadian clock.

Biological fluids contained in the interstitial space of tissues are often sampled for research and diagnostic purposes. Also it is often required that the chemical composition of the interstitial space be altered by pharmacological or physiological means. Microdialysis, which employs an invasive semipermeable membrane at the end of two open ducts makes it possible to selectively sample or deliver agents to the interstitial space. See, for example, U.S. Pat. No. 5,441,481.

The new surgical method set forth herein is a method for implantation of the guide cannula containing a microdialysis probe in the pineal. This method minimizes bleeding and eliminates or minimizes damage to pineal and non-pineal brain tissues. In addition, the method allows accurate placement of the microdialysis cannula into or next to the pineal, thus increasing the success rate tremendously. As a result, constant in vivo recovery of the pineal secretory products over long periods of time is now possible. Moreover, by integrating molecular approaches with the in vivo physiological measurement, gene regulations with their corresponding physiological consequences in single animals can be studied.

The invention is directed to a method for identifying at least one agent which modulates a preselected biological condition controlled by the circadian clock in a subject comprising a) inserting a monitoring device into, or in close proximity to the pineal, causing little or no tissue damage to the non-pineal tissue during the inserting; b) monitoring the chemical output of the pineal and monitoring a preselected biological condition of a first subject; and, c) monitoring the chemical output of the pineal and monitoring the same preselected biological condition as in step b) in a second subject after contacting the second subject with the at least one agent; wherein an alteration in the chemical output of the pineal and in the preselected biological condition in the second subject as compared to the chemical output of the pineal and preselected biological condition in the first subject identifies at least one agent which modulates a preselected biological condition controlled by the circadian clock. In one embodiment, the monitoring of the chemical output is selected from the group consisting of in vivo microdialysis and ex vivo monitoring. In a preferred embodiment, monitoring the chemical output comprises monitoring output of melatonin or serotonin (5-HT) or both.

In one embodiment of the method, the preselected biological condition is subject behavior. In a preferred embodiment of the method, the subject behavior is selected from the group consisting of symptoms of adaptation to new time zones, symptoms resulting from jet lag, symptoms of frequent shift work sleep abnormalities and symptoms of seasonal affective illnesses. In a highly preferred embodiment of the method, the symptom is selected from the group consisting of a change in hormone secretion, a change in melatonin output, a change in sleep patterns, a change in activity patterns, a change in cortisol secretion and a change in core body temperature.

In yet a different embodiment of the method, the biological condition is cellular expression of at least one biological molecule of interest. In one embodiment, the biological condition is tissue physiology.

In another embodiment of the method, the monitoring is continuous, periodic, short term, long term, or any combination thereof. In a preferred embodiment the monitoring is of a length of time sufficient to monitor one or more circadian rhythms of the subject. In one embodiment, the first subject and the second subject are the same individual.

The invention is also directed to a composition comprising one or more agents and derivatives thereof identified by the method. In one embodiment, the invention is directed to an agent or derivative thereof identified by the method in purified form. In another embodiment, the invention is directed to a pharmaceutically acceptable composition comprising one or more agents, or derivatives thereof, identified by the method.

The invention is also directed to an improved method of carrying out surgery on the pineal comprising opening the skull of a subject and inserting a monitoring device, the improvement comprising a circular dental disk drill to open the skull, and a hook to lift and/or separate nonpineal tissues away from the pineal to allow visual placement of the monitoring device into, or in close proximity to, the pineal, causing little or no tissue damage to the non-pineal tissue during the inserting. In one embodiment, the monitoring device is a microdialysis probe.

The invention is further directed to a method for implantation of a microdialysis probe for monitoring of chemicals produced by the pineal, comprising opening the skull and separating nonpineal tissue away from the pineal so as to visually expose the pineal, implanting a microdialysis probe into, or in close proximity to the pineal, causing little or no tissue damage to the non-pineal tissue during the implanting.

The invention is further directed to a method for monitoring the presence of at least one chemical in the output of the pineal comprising a) opening the skull and visually exposing the pineal; b) inserting a microdialysis probe into, or in close proximity to, the pineal, wherein non-pineal tissue exhibits little or no damage from the inserting; c) contacting the pineal or the subject with the at least one chemical; and, d) monitoring the output of the pineal for presence of the same or different chemical by in vivo microdialysis. In one embodiment, the monitoring is long term, short term, continuous or periodic or any combination thereof.

The invention is directed to a method of modulating a preselected condition controlled by the circadian clock in a subject in need thereof comprising monitoring time of onset of melatonin secretion from said subject prior to presenting a light pulse to said subject; presenting at least one light pulse to said subject, wherein said light pulse is presented during the subject's subjective night phase; and monitoring time of onset of melatonin secretion from said subject after said light pulse; wherein when said melatonin secretion exhibits a shift in the time of onset of secretion after presentation of said light pulse, said preselected condition has been modulated. In one embodiment, the light pulse is presented during the earlier half of the subjective night phase. In another embodiment, the light pulse is presented during the later half of the subjective night phase. In a different embodiment, the preselected condition is selected from the group consisting of a change in hormone secretion, a change in melatonin output, a change in sleep patterns, a change in activity patterns, a change in cortisol secretion and a change in core body temperature.

Agents

Means for in vivo or ex vivo analysis of the dialysate are known in the art. For example, the dialysate can be tested for the presence or absence of an agent, metabolite or chemical using any method known in the art of detecting that agent.

Any agent can be sampled from or delivered to the pineal. Further, the subject may be contacted in any manner with any agent, and the same agent, or a different agent, may be sampled from the pineal or any tissue. The subject may be contacted with the agent by at least one route known to those of skill in the art such as, for example, microdialysis, parenteral, subcutaneous, intravenous, intramuscular, intraperitoneal, transdermal, buccal or oral routes, either alternatively or concurrently. The dosage administered will be dependent upon the age, health, and weight of the subject, kind of concurrent treatment, if any, frequency of treatment, and the nature of the effect desired. Methods of calculating the dosage are known to those of skill in the art. Chemical output of agents, such as melatonin or serotonin, from the pineal can be determined.

Agents envisioned to be used in the practice of the invention include pharmacologically active agents, therapeutic agents, biological molecules, amino acids such as tryptophan, neuropeptides such as Substance P, mammalian tachykinins, such as, for example, neurokinin A and neurokinin B, and agonists, antagonists and derivatives of all of the above. Such agents may be administered to the subject and the chemical output of chemicals such as melatonin, for example, can be determined.

Numerous compounds employed in the art to facilitate normal sleep and to treat sleep disorders and sleep disturbances can also be delivered to the subject, and chemical output of agents such as melatonin, for example, can be sampled. These compounds include, and are not limited to, sedatives, hypnotics, anxiolytics, antipsychotics, antianxiety agents, minor tranquilizers, benzodiazepines, barbituates, beta-adrenergics, beta-blockers, compounds having a high affinity and selectivity for a serotonin receptor, and agonists, antagonists and derivatives thereof of all the above.

Various melatonin analogs, agonists, antagonists, melatonergic agents and derivatives thereof have been described in the art and can be delivered to the subject and output of agents, such as melatonin, for example, can be monitored. See, for example, U.S. Pat. Nos. 5,283,343 and 5,093,352.

Agents to be screened in the practice of the invention include, but are not limited to, compounds that are products of rational drug design, such as small molecule inhibitors, natural products and compounds having defined, undefined, or partially defined activity. An agent can be a protein-based compound, a carbohydrate-based compound, a lipid-based compound, a nucleic acid-based compound, a natural organic compound, a synthetically derived organic compound, an anti-idiotypic antibody and/or catalytic antibody, or fragments thereof. An agent can be obtained, for example, from libraries of natural or synthetic compounds, in particular from chemical or combinatorial libraries (i.e., libraries of compounds that differ in sequence or size but that have the same building blocks; see for example, U.S. Pat. Nos. 5,010,175 and 5,266,684) or by rational drug design. Many therapeutic agents are known in the art. See, for example, Remington: The Science and Practice of Pharmacy, 1995, Mack Publishing Co., Easton, Pa.

Monitoring and Screening

Using the method described herein, functions of the biological clock are assayed in depth. In addition, the method can be used for high-throughput analysis of candidate agents which may accelerate the body's adaptation to new time zones, to alleviate symptoms resulting from jet lag, frequent shift work sleep abnormalities and seasonal affective illnesses.

In many drug screening programs which test libraries of agents, compounds and natural extracts, high throughput assays are desirable in order to maximize the number of compounds surveyed in a given period of time. In a screening assay of the present invention, the subject is contacted with at least one agent of interest which may interact with any molecule of the cell, whether the cells and cellular functions are directly or indirectly, positively or negatively affected by or regulated by the agent, and the effect of the agents, compounds and extracts determined.

The changes in biological conditions which are monitored include, but are not limited to, gene transcription, protein expression, metabolic alteration, morphological alteration, lipid alteration, cell shape changes, cytoskeletal reorganization, protein translocation, protein relocalization, metal ion influx and/or efflux, changes in osmolarity, receptor expression on the cell surface, receptor clustering, receptor desensitization, protein glycosylation, protein degradation, protein phosphorylation and other protein post-translational modifications, and hormone secretion.

Changes in biological conditions such as behavior which are monitored include adaptation to new time zones, alleviation of symptoms resulting from jet lag, shift work sleep abnormalities and seasonal affective illnesses. Changes in these biological conditions are evidenced by changes in, but not limited to, hormone secretion, melatonin output, sleep patterns, activity patterns, cortisol output and in core body temperature.

Alterations in biological conditions controlled by the circadian clock can be monitored by a variety of techniques, such as, for example, in situ, in vivo or in vitro techniques. For example, alterations in the levels of various expressed proteins as determined, for example, by 2 dimensional gel electrophoresis may be compared among treated and untreated subjects in accordance with the present invention. Various methods are available for measuring, and/or monitoring, each of the conditions. For example, gene transcription may be measured by DNA chip array technology, cDNA array techniques on glass or nitrocellulose filters, oligonucleotide arrays on various solid supports, assays such as TAQman, quantitative PCR, and competitive PCR. Also included are genetic based methods of detection including fluorescence activated cell sorter analysis, fluorescence microscope analysis and antibody staining for extracellular or intracellular ligands. Such methods are known to those of ordinary skill in the art. In one embodiment, DNA chip array technology is used to measure cellular responses. For further discussion, see, for example, PCT/US00/19912.

If protein expression is the biological condition monitored, protein may be measured by methods such as mass spectroscopy, high-throughput mass spectroscopy, parallel protein identification technologies such as those based on monoclonal or polyclonal antibody recognition, HPLC, column chromatography, X-ray diffraction, and nuclear magnetic resonance spectroscopy, for example. Such methods are known to one in the art.

Once agents which modulate a biological condition are identified, the agents are further studied both in in vitro and in vivo systems. One or more of the agents of the invention may be administered alone or together. The pharmaceutical compositions may be prepared by known procedures using well-known and readily available ingredients. In making the compositions, the active ingredient will usually, but not always, be mixed with a carrier, or diluted by a carrier.

The expression or presence of any biological molecule of interest in the chemical output of the pineal may be determined by the methods of this invention. Molecules of interest are any molecules involved in any type of cellular process. Examples of molecules of interest include molecules in biochemical pathways which include, but are not limited to, those pathways for the biosynthesis of cofactors, prosthetic groups and carriers (lipoate synthesis, riboflavin synthesis, pyridine nucleotide synthesis); the biosynthesis of cell membranes, lipoproteins, surface polysaccharides, antigens and surface structures; cellular processes including cell division, chaperones, detoxification, protein secretion, central intermediary metabolism (energy production via phosphorus compounds and other); energy metabolism including aerobic, anaerobic, ATP proton motive force interconversions, electron transport, glycolysis, triose phosphate pathway, pyruvate dehydrogenase, sugar metabolism, hormone metabolism, purine and pyrimidine nucleotide synthesis, including 2′deoxyribonucleotide synthesis, nucleotide and nucleoside interconversion, salvage of nucleoside and nucleotides, sugar-nucleotide biosynthesis and conversion, regulatory functions including transcriptional and translational controls, DNA replication including degradation of DNA, DNA replication, restriction modification, recombination and repair; transcription including degradation of DNA, DNA-dependent RNA polymerase and transcription factors; RNA processing; translation including amino acyl tRNA synthetases, degradation of peptides and glycopeptides, protein modification, ribosome synthesis and modification, tRNA modification; translation factors, transport and binding proteins including amino acid, peptide, amine, carbohydrate, organic alcohol, organic acid and cation transport; and other molecules involved in the adaptation and/or specific function of the circadian clock. See, for example, U.S. Pat. No. 6,521,427. Particularly included are molecules involved in melatonin and serotonin (5-HT) production and metabolism.

Any microdialysis probe is useful in the practice of the invention. Many microdialysis probes are known in the art and envisioned in the practice of the invention. See, for example, U.S. Pat. Nos. 5,735,832; 5,441,481; 5,741,284; 5,607,391; and, 6,463,312.

In addition, any fluid can be sampled from or delivered to the pineal. Fluids envisioned to be included in the practice of the invention include cerebral spinal fluid (CSF) and any physiologically acceptable solution.

In accordance with the present invention there may be employed conventional cellular biology, biochemical, molecular biology, microbiology, and recombinant DNA techniques within the skill of the art for ex vivo monitoring. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait, ed. 1984); Nucleic Acid Hybridization [B. D. Hames & S. J. Higgins eds.(1985)]; Transcription And Translation [B. D. Haines & S. J. Higgins, eds. (1984)]; Animal Cell Culture [R. 1. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986). See also, for example, U.S. Pat. Nos. 6,337,207 and 6,333,170.

While the preferred embodiment in this invention is a surgical method described in connection with the pineal, it is understood that the surgical method has utility in other types of procedures where it is desirable to accurately position any type of microdialysis probe. Further, while another preferred embodiment of this invention is a method of identifying at least one agent which modulates a preselected biological condition controlled by the circadian clock, it is understood that the method of identifying at least one agent has utility in other types of procedures where it is desirable to monitor the chemical output of the pineal.

The invention will now be described in greater detail by reference to the following non-limiting examples.

### EXAMPLES

### Example 1

### In Vivo Microdialysis Method

Materials and Methods

Animals.

Adult Sprague Dawley male rats (220-225 g) were house at 20-25° C. with lights on at 11 am (LD 14:10). Food and were available ad libitum throughout the experiment. Illumination was supplied by white fluorescent lamps (600 lux at cage level). Rats were placed in the LD cycles for at least 2 weeks before the start of surgery.

Surgery

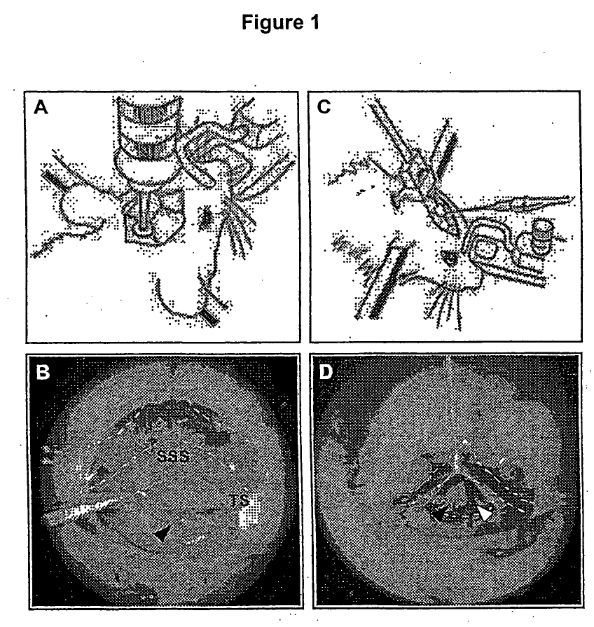

The animals were deeply anesthetized with a combination of Ketamine (10 mg/ml, 0.5 ml/100 g weight, i.p.) and Xylazine (2 mg/ml, 0.5 ml/100 g weight, i.p.). The animals' heads were shaved and positioned in a stereotaxic instrument. A longitudinal incision (approximately 2.5 cm) is made along the midline in the skin from anterior (level of the eyes) to posterior (base of the skull). The sagittal and lambdoid sutures were then exposed by scraping away the periosteum to the temporal muscles. A circular skull opening (6.8-mm in diameter), centered midline 1.5 mm posterior to the confluence of the superior sagittal and transverse sinuses, was created (FIG. 1A) using a dental disk drill equipped with a shank diamond wheel point (6.8 mm OD, Dremel, Racine USA). The dura was exposed after grinding away the top bone, which was removed using iris forceps after making a cross cut with a scalpel blade (FIG. 1B). The underlying membranes were cut open with a sharp scalpel in a T-shaped line in the area below the confluence of the superior sagital and transverse sinuses and the deflected laterally with two iris forceps (Black arrow head in FIG. 1B). Bleeding was controlled with cotton wool pledget, or flushed with water and suctioned. Three stainless steel screws were placed nearby in the parietal and frontal bones to serve as anchors. After the animal's head was repositioned 30 degrees downward, the dura matter below the confluence of sinuses was then lifted 5 mm with a hook fixed to the stereotaxic frame (FIG. 1C). The dorsal cerebellum was then pressed downward with a glass blunt tube to expose the pineal gland. The tentorium cerebelli which covers the posterior surface of the pineal and connects to the confluence of the sinuses, was then carefully reflected to visualize the pineal with a micro sharp hook, while the bleeding around the pineal was flushed and suctioned. The pineal vein is visible in the midline of the pineal posterior surface (FIG. 1D) which divides the pineal gland into two parts and drains it to the confluence of the sinuses. The tip of the guide cannula (CMA Microdialysis, N. Chelmsford, Mass.) was then positioned adjacent to either side of the exposed pineal before the skull was closed with dental cement. The rats were allowed to recover for 24 hours before experimentation.

Microdialysis and Drug Infusion

Pineal microdialysis was carried out as follows. Immediately before sampling, the rat was anesthetized with halothane briefly, the stylet (or dummy probe) was replaced with a microdialysis proble (CMA12, 20 KD cut off, membrane length 4 mm)(CMA Microdialysis, N. Chelmsford, Mass.) and fixed with plastic glue. The dialysis probe was continuously infused via the microbore PEEK tubing (0.65 mm OD, 0.12 mm ID) at a flow rate of 2 ul/min with artificial CSF (Harvard, Holliston, Mass.). Samples were collected at 10 minutes intervals via the PEEK tube into a 20 ul loop of Pollen 8 automatic injector (BAS, West Lafayette, Ind.), which was on-line with the HPLC system. The sample loop was set to be retained in the load position during the 10 minute cycles and was automatically switched to the injection positions briefly, after which the cycle was repeated. The rats were linked to the apparatus for dialysis through a quartz dual channel swivel (Harvard/Instech, Boston, Mass.) to prevent the tubing from entanglement. All pharmacological agents with molecular weights less than 6,0000 Daltons were delivered to the pineal through the microdialysis tubing. All drugs were infused after dissolving in artificial CSF solution at final concentrations indicated and for the duration described in the figure legends.

HPLC Analysis

The analytical condition for the simultaneous detection of 5-HT/NAS/melatonin was based on Drijffiout et al. (J. Neurochem. 61: 936-942 (1993)) with minor modifications. A Shimadzu pump (Shimadzu, Columbia, USA) was used in conjunction with a Shimadzu fluorescence detector (FD, excitation: 280 nm, emission: 345 nm). Samples were injected into the system through a Valco injection valve with BAS controller and subsequently separated on a reserved phase C 19 column (Supelco, 250×4.6 mm), set at a constant temperature of 30° C. using a Shimadzu column heater controlled by a Shimadzu system controller. The mobile phase consisted of a mixture of 10 mM sodium acetate, adjusted to a pH of 4.5 with concentrated acetic acid, 0.01 mM Na₂ EDTA, 500 mg/l heptane sulfonic acid and 22% (v/v) acetonitrile. The flow rate of the HPLC pump was set at 1.5 ml/min/throughout the experiment. Standard solutions were used to calibrate the system. The detection sensitivity of the fluorescence detector was set at the lowest setting to allow the full estimation of the robust serotonin rhythms. When the daytime melatonin level was important, the detector was set at its highest setting to allow consistent measurement of low levels of day melatonin secretion. The automated control of the HPLC system, the programming of the flow rate, as well as handling and storage of the chromatogram was done with an external computer using the Shimadzu Class—VP 5.03 chromatography software.

Pineal In Situ Analysis

After infusion of drugs into the pineal, animals were sacrificed by rapid decapitation and the pineals were frozen in Optimal Cutting Temperature Compound (OCT)(Sakura Fine Technical Co., Ltd., Tokyo, Japan) and sectioned using a cryostat. Care was taken to keep the microdialysis probe membrane tip in the pineal when pineals were frozen in OCT compound to aid identification of probe locations. In situ hybridization was carried out as described (Borjigin et al., J. Neurosci. 19: 1018-1026 (1999)). Briefly, digoxigenin (DIG)-labeled NAT or TPH (tryptophan hydroxylase, the rate-limiting enzyme in serotonin (5-HT) production) probes were hybridized with the fixed pineal sections overnight at 65° C. After washing, the specific signal was visualized by incubating the sections with alkaline phosphatase coupled anti-DIG antibody (Roche Molecular Biology Biochemicals, Indianapolis, USA) followed by chromatographic detection.

Results

Stable Recover of Melatonin and 5-HT Over Long Time Period

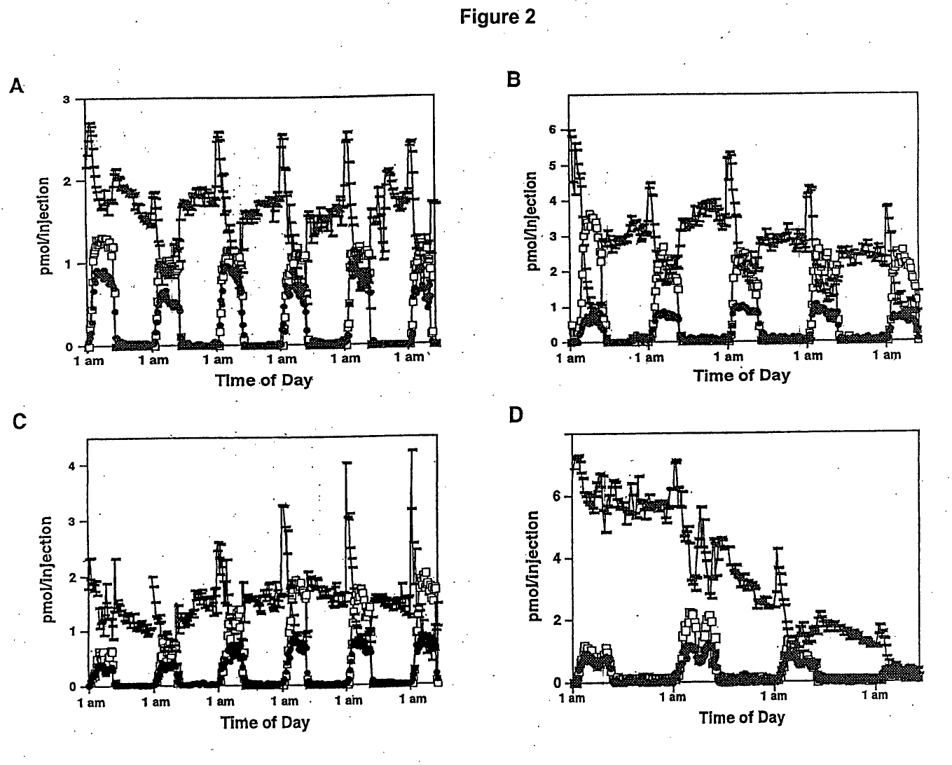

Surgical operations were performed on about 160 rats thus far, 10% (n=15) of which died during or soon after surgeries before any microdialysis data could be obtained. Of those surviving surgeries, another 10% (n=14) were judged not suitable for studies due to misplacement of the probes (assessed by visual inspection of probe locations after animals were sacrificed). A total of 114 rats gave robust circadian secretion of pineal indoles, which include serotonin (5-HT), NAS and melatonin. Secretory profiles of these rats can be divided into 4 classes, as shown in FIG. 2. Class I (FIG. 2A) includes rats with stable serotonin (5-HT) and melatonin release patterns night after night throughout the microdialysis period (some up to 4 weeks), and 59 rats (51%) belong to this class. Stable recovery refers to the consistent peak height for serotonin (5-HT) during early night was well as the average daytime secretion with little or not day to day variations. Class II (FIG. 2B) animals include 38 rats (33%) with stable melatonin secretion patterns but unstable serotonin (5-HT) in the initial 1-2 days of microdialysis. There are 7 rats (8%) that belong to the class III (FIG. 2C) which reach stable conditions for both serotonin (5-HT) and melatonin after the initial 2-4 days of microdialysis. Class IV (FIG. 2D) animals include rats whose serotonin (5-HT) and melatonin recoveries deteriorate over the entire course of the microdialysis and 9 animals (9%) were found to be in this class. Overall, the secretion patterns of serotonin (5-HT), NAS and melatonin are remarkably consistent within single animal from day to day once the recoveries are stabilized and melatonin recovery is relatively more stable compared with serotonin (5-HT) and NAS. Serotonin (5-HT) release, as evident in all the traces shown in FIG. 2, reveals a remarkable triphasic pattern: a constant day level (Phase I), a sharp increase soon after lights-off (Phase II), and a steep decline for the rest of the night period (Phase III). The detailed mechanistic analysis of the tri-phasic serotonin (5-HT) rhythm is provided elsewhere (Sun et al., Proc. Nat. Acad. Sci. USA 99: 4686-4691 (2002)). The average (+/−SEM) night values for melatonin (n=105, class I-III) and NAS (n=105, Class I-III) were 505+/−245 and 1310+/−660 fmole/injection respectively. The average values for serotonin (5-HT) (n=105, class I-III) were 1805+/−925 fmol/injection in phase 1,2880+/−1330 fmol/injection in Phase II and 945+/−495 fmol/injection in phase III. The percent increase of serotonin (5-HT) at the light-dark transition is about 150% to 200% of the daytime average. The serotonin (5-HT) secretion during the second half of the night is about half of the daytime levels.

Reproducible Effect of Isoprenaline (ISO) Stimulation of the Pineal Over Long Time Periods

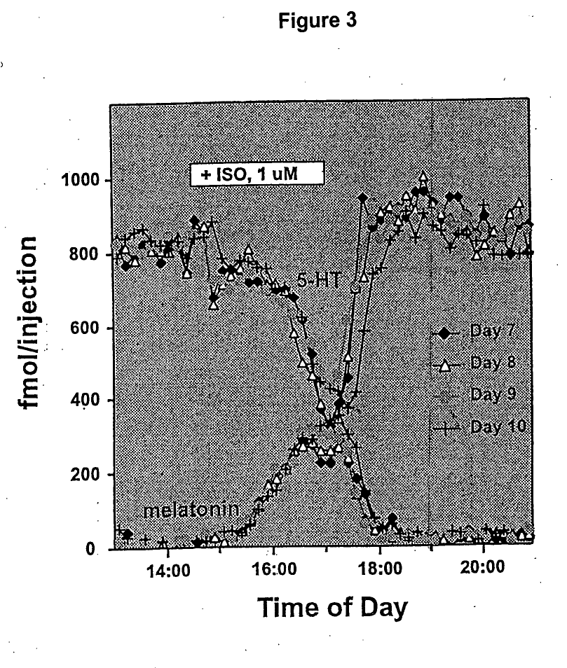

Isoprenaline (ISO) is a potent beta-adrenergic receptor agonist, which activates NAT transcription (Borjigin et al., (1999); Roseboom et al., Endocrinology 137: 3033-3045 (1996)) and melatonin synthesis (Borjigin et al., 1999; Klein et al., 1997). Pineal gland was infused with ISO (1 uM) for 2.5 hours during the day (14:30 to 17:00) through dialysis membrane on day 7 of on-line microdialysis (solid black diamond, FIG. 3). The same experiment was repeated for 3 additional days at the same time each day (14:30-17:00) in the same animals (FIG. 3, Day 8, 9 and 10). The result is remarkably consistent from day to day. As shown in FIG. 3, melatonin levels begin to increase 1 hour after infusion, reaching a maximum 2 hours after ISO treatment. Melatonin induction by ISO is elevated so long as the drug is present, and decreases rapidly within 1 hour of ISO withdrawal. As melatonin output increases, the precursor serotonin (5-HT) decreases in a perfectly inverse manner. The repeated ISO infusion of the same pineal gland during different cycles results in identical patterns of serotonin (5-HT) and melatonin secretion.

Molecular Understanding of Gene Regulation Using Integrated In Vivo Microdialysis Approaches

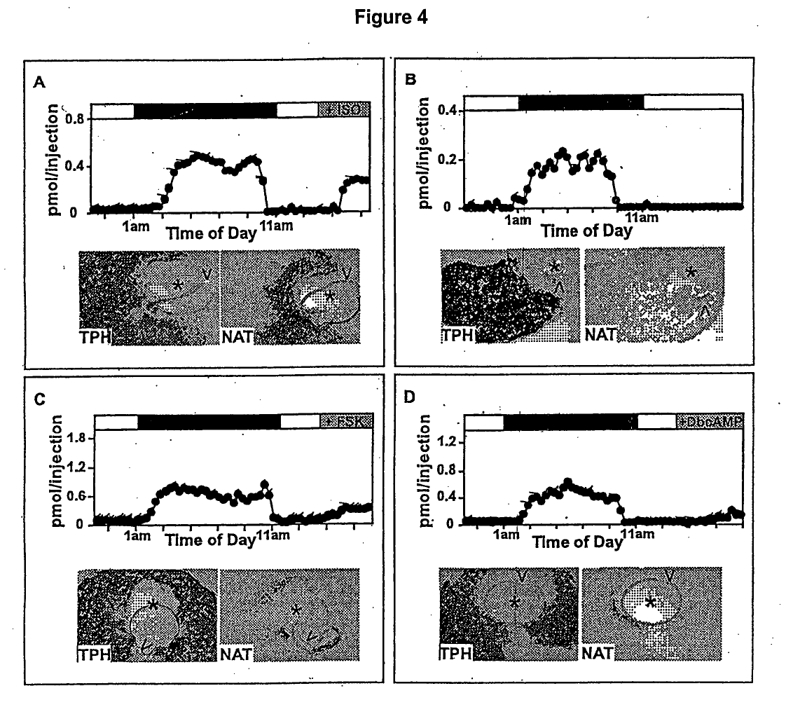

One of the most important features of in vivo microdialysis is its ability to directly influence the local cellular signaling by infusing drugs through microdialysis tube. It was sought to determine if ISO activates melatonin production by direct stimulation of NAT RNA transcription in vivo, and to examine the extent of ISO influence. On the 12th day of microdialysis of the rat shown in FIG. 3, ISO (1 uM) was infused into the pineal through the dialysis membrane at 14:00 hours. Microdialysis was terminated at 17:30 hours and the rat was sacrificed immediately for pineal in situ analysis (FIG. 4A). As a control, rat pineals microdialyzed with no exogenously added drugs were processed for in situ analysis (FIG. 4B). Consecutive sections were hybridized with TPH (lower left panel) and NAT (lower right panel) antisense RNA probes. Unlike the uniform TP RNA distribution, the ISO stimulated NAT RNA expression is restricted to the region surrounding the probe membrane (FIG. 4A). This finding is reproducible in all rats examined (n=5).

To confirm and extend the in vitro findings that forskolin and cAMP activate melatonin production by stimulating NAT gene transcription, (Borjigin et al., 1999, Roseboom et al., 1996)) forskolin and dibutyryl-cAMP were infused through the microdialysis tube during the day. The pineals were processed for in situ hybridization immediately following the stimulation of melatonin production. As shown in FIGS. 4C and 4D, in each case, the NAT gene induction is detected when melatonin production is activated and is found in a restricted ring surrounding the probe membrane. These results indicated that cAMP is the key second messenger for transcriptional activation of NAT in vivo, and demonstrate that microdialysis technique is a powerful tool to understand gene regulations in vivo when combined with molecular analysis.

Discussion

These studies demonstrate the potential of microdialysis (Arendt, 1995) to monitor pineal secretory patterns over long periods of time and (Borjigin et al., 1999) to analyze secretory patterns from a single animal with repeated stimulation and (Borjigin et al, 1995) to integrate physiological approaches with molecular analysis in a single animal. The data demonstrates the feasibility and the power of intra-subjects approach for in vivo microdialysis studies where each animal is experimented under more than one condition, thereby serving as its own control (Robinson et al., 1991). This approach opens new possibilities for in vivo analysis of pineal circadian rhythms at the molecular level and may be generally applicable to other systems.

Although stable and consistent recovery of substances over long periods of time offers tremendous advantages over the short term microdialysis, the feasibility of long term in vivo microdialysis has been debated since the development of the in vivo microdialysis (Ungerstedt, 1991). In the case of long and stable microdialysis, fewer animals are needed and the labor intensive surgery can be minimized. More importantly, a stable baseline recovery of substances allows intra-subject analysis of endogenous patterns of release and permits reliable estimation of effects of pharmacological agents within the same animals.

Two alterations from conventional surgical procedures are the key elements for success in long term pineal microdialysis. First is the use of the circular dental disk drill for opening the skull, which minimizes bleeding and damage to the underlying brain tissues and blood vessels. Second is the construction of a flexible hook that lifts the confluence of the sinuses, which exposes the pineal gland for precise targeting of the cannula, avoiding damage to the blood vessels. The usage of the hook also frees the surgeon's hands for better control of the procedure. More importantly, since the midline probe touches only the pineal gland and no other brain areas in most cases, there is very little bleeding and damage to the surrounding brain tissue.

Even with the improved surgical method, unstable serotonin (5-HT) release is still observed occasionally during the first few days of microdialysis. Similar to other microdialysis studies (Robinson et al., 1991), the extent of pineal tissue injury associated with the microdialysis probe insertion probably demonstrates the time it takes for serotonin (5-HT) to reach baseline. Prior investigator's inability to detect diurnal fluctuations of serotonin (5-HT) secretion (Drijfhout et al., Eur. J. Pharmacol. 308: 117-124 (1996)) and the reported gradual declines in the baseline levels of serotonin (5-HT) over a 24 hour period (Azekawa et al., 1991)) may have been due to irreversible tissue damage created by the surgical approach utilized in those studies.

These results also demonstrate the feasibility of using single animals to study the consequences of repeated drug infusion on pineal circadian rhythms at molecular and cellular levels. In most of the pharmacological manipulation experiments using microdialysis, independent groups of drug treated animals are used (inter group analysis). However, different animals produce different levels of melatonin and respond to pharmacological stimulants differently. Clearly, stable microdialysis with reproducible drug stimulation over long periods of time offers advantages compared to the inter-group analysis by eliminating variable in individual responses to drugs. In addition, drugs can be delivered at precise times within the circadian cycles (e.g., exactly when to administer drugs which will interfere with NAT) and single time point measurement of molecular events can be precisely timed (e.g., exactly when to sacrifice an animal to measure peak NAT mRNA). Long term in vivo microdialysis achieved in these studies opens new possibilities of integrating real time monitoring of extracellular events with molecular and cellular investigations of intracellular signal transduction, which will undoubtedly accelerate comprehensive understanding of the molecular basis of pineal circadian rhythms.

### Example 2

### Modulation of Circadian Rhythms

Although the melatonin release profile is known to be the most accurate and reliable measure of clock activity (Arendt, 1995; Lewy et al., J. Biol. Rhythms. 14: 227-236, 1999; Arendt, J. Neuroendocrinol. 15: 427-431, 2003), very few studies have been conducted in animal model systems to investigate clock functions using melatonin. The novel pineal microdialysis technique disclosed herein (Sun et al., J. Pineal Res., 35: 118-124, 2003) for automated melatonin sampling from individual animals over long periods of time has been used to conduct preliminary studies.

The studies conducted using the automated melatonin sampling technique demonstrate that (1) melatonin secretion in individual animals under entrained conditions is an extremely precise and consistent process over weeks; (2) timing of both melatonin onset (MT-on) and cessation (MT-off) maintains a unique and precise phase relationship with the light-dark (LD) cycle; (3) light induced phase shifts cause transient disturbances of the melatonin secretion pattern, whose stabilization requires restoration of the unique phase relationship with the LD cycle for both MT-on and MT-off; (4) there are large differences in the phase relationships with the LD cycle under steady state among individual animals in outbred strains; (5) with a given amount of advancement of the LD cycle, some animals display clockwise (delaying mode) shifts of melatonin secretion while others display counterclockwise (advancing mode) shifts; (6) the direction and rate of the phase shift appear to depend on the phase angle, photoperiod, strain, and perhaps, free-running period.

Circadian rhythms are found in virtually all organisms and are tightly coupled to environmental lighting conditions. A fundamental property of circadian rhythms is that they re-entrain to phase shifts of the light and dark (LD) environmental cycles (Aschoff et al., Chronobiologia, 2: 23-78, 1975). Jet lag is a common disorder which results from an inability to instantaneously synchronize our circadian rhythm to a new time zone; this can be temporarily debilitating and result in fatigue, insomnia, day-time sleepiness, dysphoria, disorientation, and gastrointestinal distress (Waterhouse et al., Lancet., 350-166-1616, 1997; Spitzer et al., Am. J. Psychiatry, 156: 1392-1396, 1999). In addition, shift work creates an instantaneous discordance between circadian and environmental rhythms. Frequent shifts of work schedules result in loss of energy, fatigue, sleep disorders, and an increased risk of cardiovascular and gastrointestinal disorders (Moore-Ede et al., The Clocks That Time Us, Cambridge: Harvard University Press, 1982; Rajaratnam & Arendt, Lancet., 358: 999-1005, 2001). Although all individuals are susceptible to such circadian disorders, it is well known that some people experience only few symptoms of jet lag and tolerate shift work schedules better, while others are troubled much more by the same circadian challenge (Ashkenazi et al., Chronobiol. Int. 14: 99-113, 1997). The remarkable heterogeneity in our biological clocks' responses to rapid shifts of the LD cycle underscores variability of basic properties of the circadian pacemaker within the normal human population.

The biological pacemaker has been studied by following various circadian rhythms driven by the clock, which include, in mammals, wheel running or locomotor activity, sleep-wake rhythms, rest-activity rhythms, temperature fluctuations, serum cortisol, urinary potassium, and serum/urinary/saliva melatonin (Moore-Ede et al., 1982; Klerman et al., J. Biol. Rhythms., 17: 181-193, 2002). Of these overt rhythms, wheel running or locomotor activity has been the most popular readout for mammalian rhythm analysis due to the ease and low cost of the procedure. Studies based on these readouts have provided enormously valuable information regarding features of circadian rhythms, including persistent free-running rhythms in constant conditions, entrainment, and temperature compensation (Johnson et al., Chronobiol. Int. 20: 741-774, 2003). Of these, the free-running rhythm and environmental light entrainment are the most well understood properties of the mammalian pacemaker.

Although studies of the circadian pacemaker using locomotor rhythms and many other behavior outputs have yielded large amounts of information, a number of studies recently have demonstrated that locomotor rhythm does not always reflect the activity of the clock and can deviate from the clock path in situations of food restriction (Kalsbeek et al., J. Biol. Rhythms, 15: 57-66, 2000) and drug use (Masubuchi et al., Eur. J. Neurosci., 12: 4206-4214, 2000). In contrast, melatonin secretion seems to be tightly associated with the clock activity even in the two situations mentioned above (Lewy, Adv. Exp. Med. Biol., 460: 425-434, 1999; Arendt, 2003). Locomotor rhythm also suffers from imprecise onset and offset of activities in some animals. These difficulties force investigators to follow the rhythms over long period of time in order to derive a statistically significant trend for both FRP and PRC. In contrast, melatonin has been shown to be the most reliable readout of the circadian clock in human studies among three different circadian markers (Klerman et al., 2002). Melatonin has therefore gained increasing popularity in human circadian rhythm studies in recent years (Lewy, 1999; Arendt, 2003). In animal model systems, however, the small blood volume in laboratory animals and the challenge of sampling frequent serum or urinary melatonin levels around the clock for many days have limited its use as a marker for biological rhythm analysis.

Free Running Rhythms

Circadian clocks have an endogenous free-running period (FRP) that is close to, but not exactly 24 hours. The FRP varies between species and differs among individuals in the same species (Pittendrigh & Daan, J. Comp. Phsyil., 106: 291-331, 1976; Aschoff, Handbook of Behavioral Neurobiology, Volume 4, biological Rhythms, New York: Plenum Press, Chapter 6, pp. 81-93, 1981). In constant conditions these rhythms free run with a defined FRP in any given individual. They can be modulated by conditions such as the photoperiod, phase angle, and experimental manipulation of the LD period (artificial T-cycles) (Aschoff, Handbook of Behavioral Neurobiology, Vol. 4, Biological Rhythms, Chapter 6, pp. 81-93, 1981; Johnson et al., 2003). Although the FRP has been measured from a variety of organisms, almost all studies in mammals utilized locomotor rhythm as a readout of the circadian clock, and the FRP has never been studied in organisms other than humans (Czeisler et al., Science, 284: 2177-2181, 1999) using melatonin as the clock marker.

Entrainment of Circadian Rhythms by Light

Circadian rhythms found in virtually all organisms oscillate with an FRP that is often not exactly 24 hours. The central task of the biological clock is to adjust or entrain the FRP precisely to the 24-hour period of the environment so that the entrained rhythm establishes a stable phase relationship or phase angle with the entraining oscillations. As clearly stated by Johnson et al., (2003), demonstration of entrainment goes beyond showing a 24 h rhythm in an LD cycle; it is necessary to show that ‘the period of rhythm equals the period of the LD cycle with a stable and unique phase angle.’ Of the entraining rhythms or zeitgeber in the environment, which include the LD cycle, temperature oscillations, humidity, cyclic food availability and social cues, the LD cycle is the most consistent environmental time cue (Aschoff & Daan, The Entrainment of Circadian Systems, Handbook of Behavorial Neurobiology, Volume 12, Circadian Clocks, New York: Kluwer/Plenum, Chapter 1, pp. 7-43, 2001; Johnson et al., 2003). Consequently most information about entrainment is derived from studies using LD cycles. The entrainment properties of the circadian clocks have never been studied using melatonin as an output in any animal model system.

Phase Response Curve (PRC)

The circadian pacemaker responds to light stimulation differently at different times (or phases) of an animal's circadian cycle, a key characteristic described by the ‘phase response curve’ (PRC), a plot of phase shifts of circadian rhythms as a function of the circadian phase that a light stimulus is presented (DeCoursey, Science, 131: 33-35, 1960; Pittendrigh, Circadian rhythms and the circadian organization of living systems, Cold Spring Harbor Symposia on Quantitative Biology, Volume 25, Biological Clocks, pp. 159-184, 1960; Aschoff, Circadian Clocks, Amsterdam: North-Hollan, pp. 95-111, 1965a; Daan & Pittendrigh, J. Comp. Phsyil., 106: 253-266, 1976; Johnson, Chronobiol. Int., 16: 711-743, 1999). Light pulses given in the subjective day (circadian time; CT 0-12) have little or no effect on the onset of activity on subsequent days, regardless of whether animals are nocturnal or diurnal. In contrast, light pulses given during the subjective night phase (circadian time; CT 12-24) shift the rhythm. Light pulses presented during the earlier half of the subjective night phase delay the overt rhythm, while those presented during the later half of the subjective night phase advance the rhythm (DeCoursey, 1960; Pittendrigh & Daan, 1976; Johnson, 1999; Johnson et al., 2003). To date, however, there has been no report of PRC derived from any animal, besides humans (Lewy et al., Chronobiol. Int., 15: 71-81, 1998; Khalsa et al., J. Physiol., 549: 945-952, 2003), that have been conducted with single light pulses using melatonin as a marker.

Various protocols are available for measurement of the PRC. The classic method of deriving the PRC is to assay the phase shift after a single light pulse delivered at predetermined circadian phases, while animals freerun in constant darkness (DeCoursey, 1960). This method of PRC determination is called the Aschoff protocol #1 (Aschoff, 1965a) and requires knowledge of the real-time behavior of the pacemaker as the experiment progresses. The Aschoff protocol #2 involves releasing animals from a LD cycle into constant conditions (such as DD) and then giving a light pulse during the first few subjective days of the free run (Aschoff, 1965a; Moore-Ede et al., 1982). Following a light pulse, overt rhythms go through several transients before reaching a new steady state, especially when the light pulse advances the rhythm. Two methods have been used for derivation of the PRC with either of the protocols described above (Moore-Ede et al., 1982): (1) the next day method (or the immediate PRC method), which measures phase shifts 24 hours after the light pulse is given; and (2) the steady state method, which measures the amount of shift after all transients are stabilized at the new steady state.

FRP and Photoperiod

Circadian rhythms can be entrained with a LD cycle possessing different photoperiods. Different photoperiods, however, can affect the FRP dramatically with a phenomenon called ‘aftereffect’ (Endo et al., Jpn. J. Physiol. 49: 425-430, 1999) upon releasing animals into constant darkness (DD). Studies with both birds (Aschoff, 1981) and mammals (Aschoff, 1981; Pittendrigh & Daan, 1976) suggest that animals entrained in an LD cycle that has a longer photoperiod will free run in DD with a shorter FRP, while those in shorter photoperiod have longer FRP in DD. The relationship of the FRP and the photoperiod has never been studied in any organisms including humans using melatonin as a marker.

FRP and Phase Angle

Endogenous circadian rhythms of different organisms, or individuals within the same species, initiate their rhythmic activities with different phase angles (Aschoff, Circadian Clocks, Amsterdam: North-Hollan, pp. 262-276, 1965b) with respect to the entraining LD cycle (either onset of dark, for nocturnal animals, or onset of light, for diurnal animals). When animals are entrained either with cyclic temperature rhythms, or with LD cycles (Aschoff et al, Comp. Biochem. Physiol. 18: 397-404, 1966) of 24 hours, those with larger phase differences between onset of activities and onset of temperature step or light onset are found to have shorter FRP. We will attempt to address the relationship between the phase angle and the FRP using melatonin as a marker.

Re-entrainment of Circadian Rhythms after Phase Shifts of the LD Cycle

While most of us are not consciously aware on a daily basis of our internal biological clocks, almost all of us come to acute appreciation of it during jet lag. After a sudden shift of the LD cycle upon crossing multiple time zones rapidly, the clock cannot adjust to the new local time zone right away (Mills et al., Ergonomics, 21: 755-761, 1978a). It usually takes several periods for the organism to become re-entrained and to again reach a steady state phase relationship (or phase angle) with the local LD cycle (Mills et al., J. Physiol. 285: 455-470, 1978b; Minors & Waterhouse, J. Biol. Rhythms, 9: 275-282, 1994). The time it takes for organism's pacemaker to reach the new steady state (duration of re-entrainment) and direction the internal clock adjustment, whether via clockwise shift (CW) or counterclockwise shift (CCW), are determined by a number of factors that include (but are not limited to) FRP, photoperiod (Aschoff et al., 1975), and perhaps the characteristics of the PRC map.

FRP and Rate and Direction of Re-Entrainment

Studies of the lizard's locomotor activity rhythm demonstrate that animals with shorter FRP shift their clocks in the CCW direction during a 9 hr-advance shift of temperature (not LD) cycle, while those with longer FRP re-entrain in a CW direction (Aschoff et al, 1975). In addition, fruit bats with longer FRP of locomotor activity re-entrain faster than those with shorter FRP in a delay shift of the LD cycle; in contrast, animals with shorter FRP reach stable re-entrainment sooner than those with longer FRP during advance shifts (Aschoff et al, 1975). As photoperiod and phase angle affect the periods of free running rhythms in constant conditions, the rate and direction of re-entrainment are also expected to change according to these parameters. To date, however, there have been few studies investigating the relationship between the re-entrainment process and the basic properties of the circadian pacemaker using melatonin as a marker.

PRC and Direction of Re-Entrainment in Animal Studies

One of the key features of the PRC is that there is a crossover point (COP) during the subjective night where single light pulses do not elicit phase shifts during subsequent cycles. Our literature survey revealed that while animals in the same strain respond to a given light pulse to a different extent (i.e., magnitude of phase shifts can be quite different among individuals of the same species)(Daan & Pittendrigh, 1976; Honma et al., Experientia., 34: 1602-1603, 1978; Summer et al., Am. J. Physiol., 246: 299-304, 1984; Honma et al., Jpn. J. Physiol., 35: 643-658, 1985), the positions of the COP in the PRC among individuals of the same species are remarkably conserved. For instance, the COP is at CT 20 for M. musculus (Daan & Pittendrigh, 1976) and CT 19:30 for Wistar rats (Honma et al., 1985), and CT 18 for Sprague Dawley rats (Summer et al., 1984). Illnerova's group have used serotonin N-acetyltransferase (NAT), the key enzyme in melatonin production in the pineal gland (Klein & Weller, Science, 169: 1093-1095, 1970; Borjigin et al., 1999), as a circadian marker for a population analysis of Wistar rat phase shift behavior (Illnerova & Humlova, Neurosci. Lett. 110: 77-81, 1990). While 5-hr advance shifting of the LD cycle induced a phase shift of NAT rhythms only in CCW directions, 7 or 8 hr advance shifts of the LD cycle elicited NAT rhythm shifts only in CW direction (Illnerova & Humlova, 1990). These results suggest there may be a tight correlation between the COP of a given PRC with the critical point of the circadian cycle when the directions of re-entrainment switch from CCW to CW. As it is obviously difficult to frequently sample melatonin from individuals over many cycles, all studies mentioned above utilized behavior or temperature output (or NAT) as markers of circadian clocks, and none of the animal studies have demonstrated both CW and CCW shifts in the same strain with a given amount of advance shift of the LD cycle.

PRC and Direction of Re-Entrainment in Human Studies

Studies of clock adjustment to new time zones have been conducted mostly in human subjects. A number of general features emerge from these and other studies: 1. As the number of time zones crossed increases, the re-entrainment time lengthens for both humans (Mills et al., 1978b) and experimental animals (Aschoff et al, 1975). 2. Phase-delays proceed more rapidly than phase advances for mammals including humans (Aschoff et al, 1975; Eastman & Martin, Ann. Med., 31: 87-89, 1999) with a given amount of phase shift. 3. When a LD cycle is advanced (CCW direction, or as in eastward travel) for more than 5 hours, humans can adjust their endogenous clocks in two ways: a CCW shift that follows the direction of the entraining LD cycle shift; or a CW shift (or antidromic) that goes the opposite direction of the LD cycle shift (Elliot et al., J. Physiol. 221: 227-257, 1972; Aschoff et al., 1975). 4. Advancing larger numbers of time zones is more likely to cause a CW shift (Aschoff et al., 1975; Gundel & Wegmann, Chronobiol. Int., 6: 147-156, 1989). A human PRC generated by single pulses of light (Minors et al., Neurosci. Lett., 133: 36-40, 1991; Khalsa et al., 2003) suggests that the COP in humans is close to the time of minimal body temperature during the subjective nighttime and close to the critical point where re-entrainment to advancing the LD cycle shifts from CCW-advancing to CW-delaying mode.

Long Term Sampling of Circadian Melatonin Output in Freely Moving Individual Animals

Long term sampling of melatonin from individual animals has been difficult due to the small volume of blood in animal model systems. Moreover, the rate of changes of human serum/urine/saliva melatonin may not directly reflect the changes that take place in the pineal gland, due to a finite half-life of melatonin in the serum and metabolism of melatonin in liver. The newly invented surgical technique disclosed herein enables us to frequently and accurately measure melatonin secretion directly from the pineal gland over many circadian cycles.

High Resolution Mapping of Pacemaker Function

Previous studies utilizing melatonin in humans or NAT, the key enzyme for melatonin synthesis, as a marker in animals (Illnerova's group) were performed using infrequent sampling (hourly or longer intervals), limiting their interpretation to low temporal resolution. The microdialysis approach discussed herein allows sampling of melatonin as frequently as every 10 min, which permits an unprecedented, detailed analysis of pacemaker function.

Automated Mapping of Pacemaker Function

Most studies reported in the literature using melatonin as an output use either ELISA or radioimmunoassay to estimate the melatonin concentrations in the collected samples (Arendt, 1995), which is labor intensive and error prone. In contrast, the approach we utilized allows automated analysis that bypasses manual handling of the sample at all times, which reduces labor and errors.

Real-Time Monitoring of Pacemaker Activity

Data published in current studies utilizing melatonin as a marker are mostly analyzed a few hours or days after the studies are concluded, which do not permit real time adjustment of study protocols. Our approach, in contrast, allows real-time investigation of the pacemaker function and permit appropriate real-time adjustments of experimental conditions. The series of preliminary findings below are part of studies initially intended to analyze the re-entrainment processes.

An FRP for Melatonin Secretion

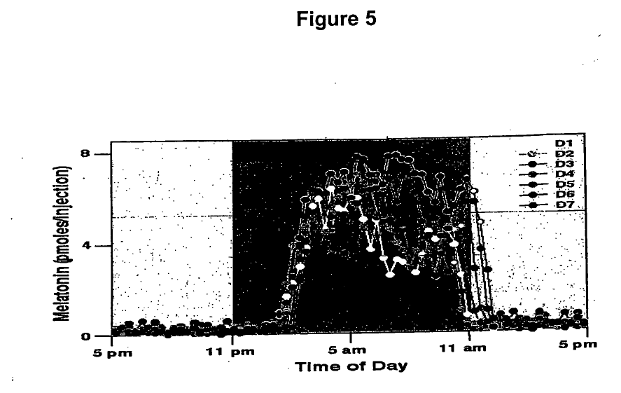

To derive FRP, we released rats (Sprague Dawley-SD, male) after a period of entrainment at LD12:12 (12 hours of photoperiod) into constant darkness (DD) and followed melatonin secretion (FIG. 5). In this and all subsequent experiments, the melatonin onset (MT-on) is defined by the time when melatonin increased more than 2-fold compared to day levels (black-filled circles); melatonin offset (MT-off) is defined by the last data point of the melatonin peak immediately before the decline from its nocturnal levels (white-filled circles). Under the entrained conditions (FIG. 5, data from the first 3 days is shown for a typical rat) MT-on occurs at 1:20 am, and the MT-off at 10:20 am. This phase relationship (or phase angle) to the entraining LD cycle (lights on at 11 am, off at 11 pm) is maintained on day 1-3, which indicates a stable entrainment. Both the MT-on and the MT-off display a consistent delay shift of 20 min per day (except for MT-on on day 5, which stalled for one cycle) upon release to DD and a free-run with a period of 24.33 hrs per cycle (calculated using MT-off). An increase of 20 min in melatonin secretion duration is noted after the transition from LD to DD, which is possibly due to the decompression effect reported by others (Hastings et al., J. Endocrinol., 114: 221-229, 1987; Illnerova & Vanecek, Brain Res., 261: 176-179, 1983; Illnerovaetal., Brain. Res. 362: 403-408, 1986; Puchalski & Lynch, Am. J. Physiol. 261: 670-676, 1991). Photoperiod changes are known to affect the duration of melatonin secretion in seasonal reproductive animals (Arendt, 1995). It may also affect the experimental estimation of the FRP of the melatonin rhythm in DD, if this occurs in the strains of rats used in our experiments, as decompression of NAT activity duration was observed in Wistar rats upon shifting from long to short photoperiods (Illnerova & Vanecek, 1983; Illnerova et al., 1986). For the rat shown in FIG. 5, 9.33 hrs of secretion duration in DD may represent the natural melatonin activity under relaxed conditions, and may vary among individual rats depending on the phase angle difference with the entraining LD cycle (see below). This set of experiments also demonstrates that phase estimates using melatonin, when a combination of MT-on and MT-off is used, can dramatically improve the accuracy and accelerate the pace of rhythm studies (in contrast to the many weeks of constant conditions that are often required for behavior output analysis).

Photoperiod

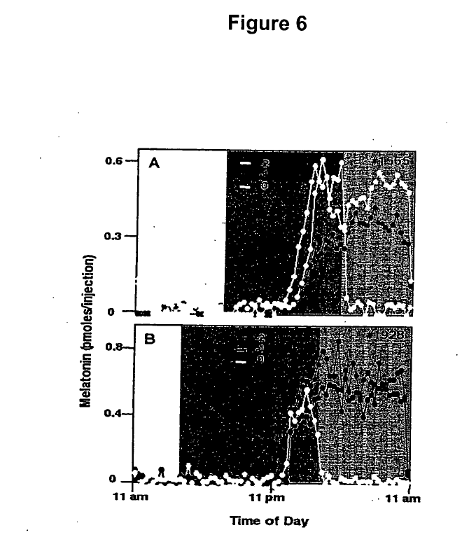

In many animals, the total time of melatonin secretion increases when the night period increases. The more compressed the melatonin secretion is in the long photoperiod, the larger the decompression effect there should be following release, as demonstrated with photoperiodic animals (Hastings et al., 1987). Decompression of melatonin can occur by either advancing MT-on or delaying MT-off. The direction of decompression may depend on the period of the endogenous pacemaker. We have given PVG rats a 6 hr-advance shift of an LD14: 10 cycle (FIG. 6A) and an 8 hr-advance shift of an LD12: 12 cycle (FIG. 6B). Both shifts are accomplished by lengthening the dark periods (darker shades). On the day of the shift, there is already an advance shift of 40 min of MT-on (white tracing in FIG. 6A). Interestingly, the same amount of shift is never seen in any of the animals (8 PVG rats) we tested on LD 12:12 (see the white tracing in FIG. 6B). The advance shift of 40 min in rat #1565 on day 0 (seen in 3 out of 3 rats tested, not shown) is interpreted to represent the transient decompression of MT-on upon release of the inhibitory constraint of the previous LD transition, which may be in a relaxed state already under the shorter photoperiod of 12 hours. An alternative explanation is that the rat #1565 has a much shorter FRP, which ‘free’ runs in the expanded darkness.

Phase Angle Variability

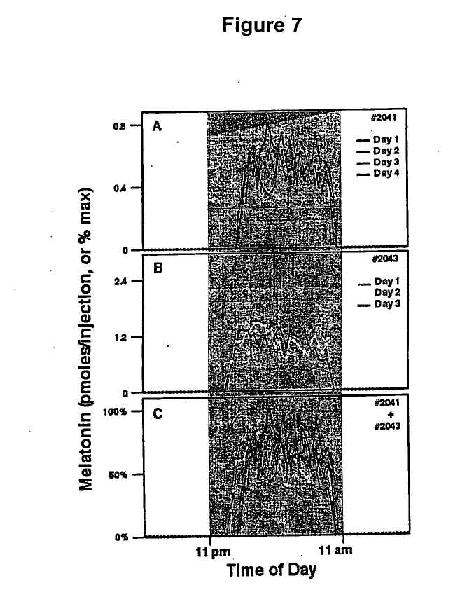

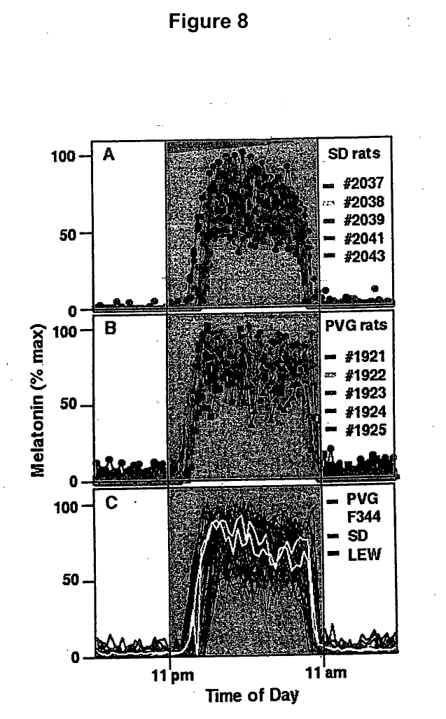

Phase relationships of MT-on and MT-off with the entraining LD cycle vary among individuals in the same strain and vary greatly between strains of rats under entrained conditions. Four (rat #2041) and three (rat #2043) consecutive days of melatonin secretion collected from two SD rats are shown in FIG. 7. Within the same animal, melatonin secretion is an extremely precise process from day to day under entrained conditions, where both MT-on and MT-off maintain stable phase angles in relation to lights-off (for MT-on) and lights-on (for MT-off) (7A and 7B). On the other hand, two individual rats may display different phase angles (FIG. 7C). There is a 1 hr difference in the timing of MT-on between #2041 and #2043, and yet the timing of MT-off is indistinguishable between the two individuals. This difference is larger when 4 strains of rats are compared (FIG. 8). Data from 2-4 consecutive days are shown for each rat from both SD (FIG. 8A) and PVG (FIG. 8B) strains. Compared to the larger variations of MT-on in the SD strain (range from 12:40 am to 1:40 am; MT-off ranges from 9:40 am to 10:40 am between rats, see FIG. 8A), PVG rats showed smaller fluctuations between members (MT-on from 12 am to CT 12:40 am; and MT-off from 10:20 am to 10:40 am, FIG. 8B). The smaller inter-individual differences are expected, as PVG rats are inbred compared to the SD rats, which are outbred animals. When data from all rats (over several days each) from 4 different strains are compared (6 rats from PVG, 5 from SD, 2 (2 cycles each) from F344, and 2 (1 cycle each) from LEW), a clearer trend emerges (FIG. 8C). Melatonin secretion from LEW rats has the shortest duration of 8 hrs, while PVG rats last for about 10 hrs. The phase differences are more pronounced for MT-on, which is, on the average, 12:20 AM for PVG, 12:40 AM for F344, 1 AM for SD, and 2 AM for LEW. Whether these differences are upheld in DD remains to be seen.

Rate of Phase Shift of Pacemaker to a New Time Zone

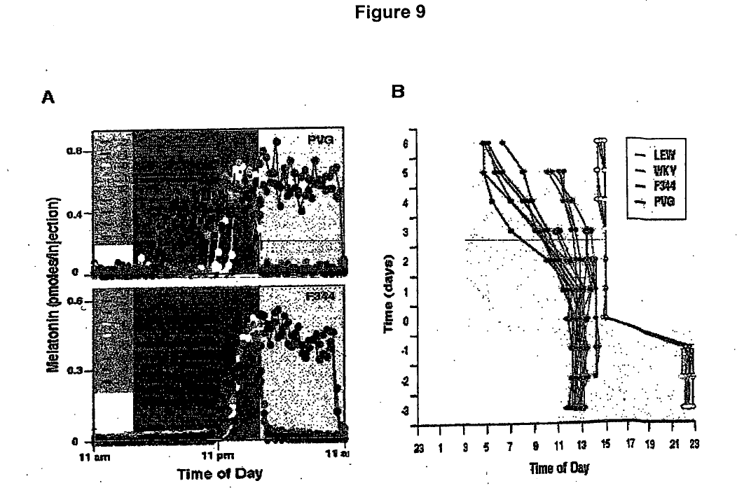

The large variation in phase angles presented above prompted us to test the relation of the phase angle to the rate of adjustment to a time zone shift. Animals were given (entrained in LD12:12 for more than a month) an 8 hour-advance shift of the LD cycle, and their melatonin output was followed. All shifts are accomplished by lengthening the dark period. Melatonin secretion on the day of the shift (Day 0) is marked by the white tracing. As shown in FIG. 9A, an 8 hr-advance shift of the LD cycle caused a shift of melatonin secretion in the CCW direction for PVG and F344 rats, which is the direction of the LD cycle change. The rate of shift, however, is markedly different in the two strains of rats. While PVG rats shifted 6 hours in 5 days (about 1.2 hours per day), the F344 rats moved only one hour in 5 days (12 min per day). Although F344 rats have a slightly shorter duration of melatonin secretion (see above) compared to PVG rats and a slightly later MT-on, the small difference in phase angle cannot account for the dramatic variations in the rate of shift, especially considering how LEW rats shifted (FIG. 9B). In this diagram, we plotted both MT-on (black filled circles) and MT-off (white circles) during the course of the experiment for PVG (6 rats), WKY (5 rats), LEW (4 rats) and F344 (4 rats). Again, within each inbred strain of rats, the phase angle and rate of shift were highly consistent. However, while LEW rats shifted about 20 min in 2 days, there was no movement of the pacemaker in F344 rats whose phase angle is between those of LEW and PVG rats. Clearly, phase angle alone cannot explain the large discrepancy seen in this experiment.

Rate of Re-Entrainment of the Pacemaker

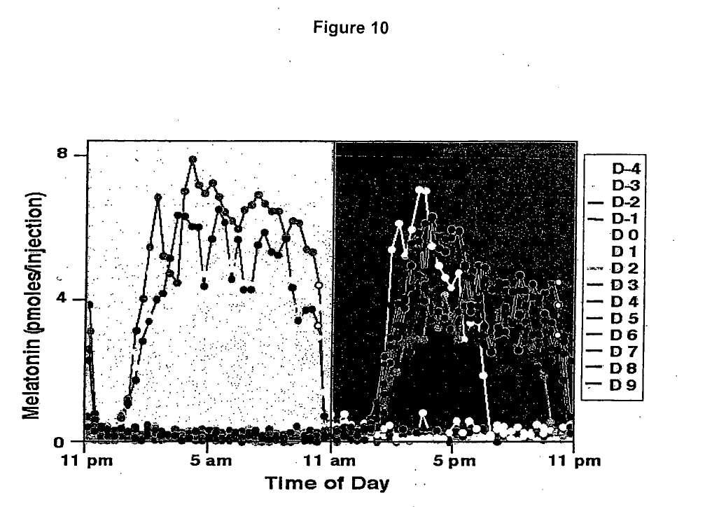

The experiments shown in FIG. 9B demonstrate that rats re-entrain with varying rates following a shift of the LD cycle. Here, we define completion of re-entrainment as when a stable and unique phase angle is restored after completion of a phase shift. Data shown in FIG. 10 examines a single, representative rat whose re-entrainment was accomplished much later than the completion of the phase shift; this is evident only in high-resolution pineal microdialysis studies which show that MT-off fails to precede the DL transition until day 7 after the time shift. In these experiments, rats (1 out of 5 rats is shown) were exposed to a reversed LD 12: 12 cycle, by lengthening the dark period for 12 hours. As shown in FIG. 10, the pacemaker of the rat is at an entrained state with MT-on at 1:20 am, and MT-off at 10:20 am from day −4 to day −1. Following reversal of the LD cycle, melatonin was secreted at night, and continued to expand toward the new DL transition (where lights-on occurs) on the subsequent cycles. During the entire shift, MT-on appeared to be locked in the phase position from the very beginning. In contrast, MT-off shifts gradually on D1 and D2, then overshoots on D3 (remaining at the junction of the DL transition as if it was terminated by the premature onset of light. Only after another 4 days does the pacemaker restore MT-off to the proper phase angle (D7, red tracing). These data demonstrate that the re-entrainment process of any given pacemaker may be composed of two stages: a phase dependent shift and finer adjustment of the phase, which takes a longer time than the phase shift itself.

Direction of Re-Entrainment of the Pacemaker Following a Time Zone Advance

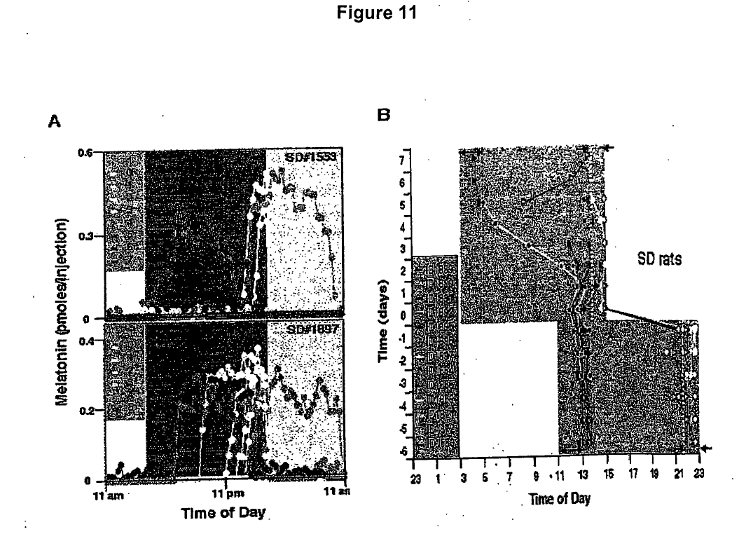

An 8 hr-advance shift of the LD cycle is known to cause some pacemakers to re-entrain in the CW (antidromic) direction (Aschoff et al., 1975; Illnerova & Humlova, 1990), a phenomenon observed in human studies as well (Aschoff et al., 1975; Minors et al., Chronobiol. Int. 11: 356-366, 1994; Minors & Waterhouse, 1994). Our studies indicate that there are large strain differences in pacemaker operation under these conditions. In contrast to published studies, none of the 4 strains of inbred rats (PVG, F344, WKY, and LEW) displayed CW shifts (FIG. 9B). However, SD rats demonstrated a range of pacemaker preferences for the rate and direction of re-entrainment when given the same experimental LD advancement. #1553 (11A; upper panel) made no move on day of the shift (Day 0), delayed its onset (instead advancing) for 40 min one day after the LD shift, disappeared for 3 days (due to suppression by light-masking, Aschoff, Jpn. J. Physiol. 49: 11-18, 1999), acquired the proper MT-on phase angle (with the new LD cycle) on day 5, and then finally began shifting its MT-off. Rat #1897 (11A; lower panel), in contrast, phase advanced on the day of the shift as soon as the dark period was expanded, and advanced further with an average rate of 2 hrs per day. Although these two rats differ in their phase angle especially for MT-off (10:00 for #1553, and 10:40 for #1897), for reasons discussed above (see FIG. 9C) and as presented in FIG. 11B, their dramatically different adjustments to the same LD cycle change cannot be explained only by the phase angle difference. Data for 13 SD rats are summarized in FIG. 11B. One (#1897) of the 13 rats proceeded in CCW direction rapidly and almost completed re-entrainment 5 days after the shift, while 4 rats showed very little movement of their clocks for 2 days (#1869), 3 days (#1865), and even 5 days (#1890 and #1895) days after the LD cycle shifts. Eight of them proceeded in a CW direction on one day after the shift (quickly displaying no detectable melatonin), one of which (#1553, see FIG. 11A, upper panel) jumped forward, while others continuously drifted into the light period and thus had undetectable melatonin. Some general features emerge from these data (and others not presented here). (1) Once the pacemaker shifts in the CW direction, it stays with that course and never reverses direction before the shift is complete. (2) The pacemaker irreversibly commits to a direction early in the process, usually within 24 hours after the shift. (3) The pacemakers that make very little progress in their re-entrainment during the first two cycles always shift in the CCW direction. These rhythmic behaviors have rarely been described in the literature and open a window of opportunity for a mechanistic dissection of the pacemaker's operation.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

All cited patents, patent applications and publications and other documents cited in this application are herein incorporated by reference in their entirety.

### REFERENCES

Arendt (1995) Melatonin and the Mammalian Pineal Gland. London: Chapman & Hall.
Arendt (2003) Importance and relevance of melatonin to human biological rhythms. J. Neuroendocrinol. 15: 427-431.
Aschoff (1965a) Response curves in circadian periodicity. In: Aschoff, J., ed. Circadian Clocks. Amsterdam: North-Hollan, pp. 95-111.
Aschoff (1965b) The phase angle difference in circadian periodicity. In: Aschoff, J., ed. Circadian Clocks. Amsterdam: North-Hollan, pp. 262-276.
Aschoff et al. (1966) Circadian period and phase-angle difference in chaffinches (Fringilla coelebs L.). Comp. Biochem. Physiol. 18: 397-404.
Aschoff et al. (1975) Re-entrainment of circadian rhythms after phase-shifts of the Zeitgeber. Chronobiologia. 2: 23-78.
Aschoff (1981) Freerunning and entrained circadian rhythms. In: Aschoff, J., ed. Handbook of Behavioral Neurobiology. Volume 4. Biological Rhythms. New York: Plenum Press, Chapter 6., 81-93.
Aschoff (1999) Masking and parametric effects of high-frequency light-dark cycles. Jpn J. Physiol. 49: 11-18.
Ashkenazi et al. (1997) Interindividual differences in the flexibility of human temporal organization: pertinence to jet lag and shiftwork. Chronobiol Int. 14: 99-113.
Azekawa et al., (1991) Pineal microdialysis in freely moving rats, Brain Res. Bull. 26: 413-417.
Azekawa et al. (1991) Diurnal changes in pineal extracellular indoles of freely moving rats, Neurosci. Lett. 132: 93-96.
Ausubel et al. (eds.) (1994) Current Protocols in Molecular Biology, John Wiley & Sons, Inc.
Borjigin et al. (1995) Diurnal variation in mRNA encoding serotonin N-acetyltransferase in pineal gland. Nature. 378: 783-785.
Borjigin et al. (1999) A novel pineal night-specific ATPase encoded by the Wilson disease gene. J. Neurosci. 19: 1018-1026.
Borjigin et al. (1999) The pineal gland and melatonin: molecular and pharmacologic regulation. Ann. Rev. Pharmacol. Toxicol. 39: 53-65.
Borjigin et al.(2002) Treatise on Pineal Gland and Melatonin, Science Publishers, Inc., Enfield, 423-430.
Calos (ed.) (1987) Methods In Enzymology, Volume 154, Cold Spring Harbor Laboratory).
Czeisler et al. (1999) Stability, precision, and near-24-hour period of the human circadian pacemaker. Science. 284: 2177-2181.
Daan & Pittendrigh (1976) A functional analysis of circadian pacemakers in nocturnal rodents. II. The variability of Phase Response Curves. J. Comp. Phsyil. 106: 253-266.
Daan & Aschoff (2001) The Entrainment of Circadian Systems. In: Takahashi, J. S., Turek, F. W., Moore, R. Y., ed. Handbook of Beharioral Neurobiology. Volume 12. Circadian Clocks, New York: Kluwer/Plenum, Chap. 1, pp. 7-43.
DeCoursey (1960) Daily Light Sensitivity Rhythms in a Rodent. Science. 131: 33-35.
Drijfhout et al. (1993) Microdialysis of melatonin in the rat pineal gland: methodology and pharmacological applications. J. Neurochem. 61: 936-942.
Drijfhout et al. (1996) Parasympathetic inhibition of pineal indole metabolism by prejunctional modulation of noradrenaline release. Eur. J. Pharmacol. 308: 117-124.
Eastman & Martin (1999) How to use light and dark to produce circadian adaptation to night shift work. Ann Med. 31: 87-98.
Elliot et al. (1972) The effect of real and simulated time-zone shifts upon the circadian rhythms of body temperature, plasma 11-hydroxycorticosteroids and renal excretion in human subjects. J. Physiol. 221: 227-257.
Endo et al. (1999) After-effect of entrainment on the period of human circadian system. Jpn J. Physiol. 49: 425-430.
Freshney (ed.) (1986) Animal Cell Culture.
Gait (ed.) (1984) Oligonucleotide Synthesis.
Glover (ed.) (1985) DNA Cloning: A Practical Approach, Volumes I and II.
Gundel & Wegmann (1989) Transition between advance and delay responses to eastbound transmeridian flights. Chronobiol. Int. 6: 147-156.
Haines & Higgins (eds). (1984) Transcription And Translation.
Hames & Higgins (eds.) (1985) Nucleic Acid Hybridization.
Hastings et al. (1987) Effect of asymmetrical reductions of photoperiod on pineal melatonin, locomotor activity and gonadal condition of male Syrian hamsters. J. Endocrinol. 114: 221-229.
Honma et al. (1978) A phase response curve for the locomotor activity rhythm of the rat. Experientia. 34: 1602-1603.
Honma et al. (1985) Response curve, free-running period, and activity time in circadian locomotor rhythm of rats. Jpn J. Physiol. 35: 643-658.
Illnerova & Vanecek (1983) Extension of the rat pineal N-acetyltransferase rhythm in continuous darkness and on short photoperiod. Brain Res. 261: 176-179.
Illnerova et al. (1986) Adjustment of the rat pineal N-acetyltransferase rhythm to change from long to short photoperiod depends on the direction of the extension of the dark period. Brain Res. 362: 403-408.
Illnerova & Humlova (1990) The rat pineal N-acetyltransferase rhythm persists after a five-hour, but disappears temporarily after a seven-hour advance of the light-dark cycle: a six-hour shift may be a turning point. Neurosci. Lett. 110: 77-81.
Immobilized Cells And Enzymes (1986) IRL Press.
Johnson (1999) Forty years of PRCs—what have we learned? Chronobiol. Int. 16: 711-743.
Johnson et al. (2003) Entrainment of circadian programs. Chronobiol. Int. 20: 741-774.
Kalbeek et al. (2002) Restricted daytime feeding attenuates reentrainment of the circadian melatonin rhythm after an 8-h phase advance of the light-dark cycle. J. Biol. Rhythms. 15: 57-66.
Khalsa et al. (2003) A phase response curve to single bright light pulses in human subjects. J. Physiol. 549: 945-952.
Klein & Weller (1970) Indole metabolism in the pineal gland: a circadian rhythm in N-acetyltransferase. Science. 169: 1093-1095.
Klein et al. (1997) The melatonin rhythm-generating enzyme: molecular regulation of serotonin N-acetyltransferase in the pineal gland. Recent Prog. Horm. Res. 52: 307-357; discussion 357-358.
Klerman et al. (2002) Comparisons of the variability of three markers of the human circadian pacemaker. J. Biol. Rhythms. 17: 181-193.
Lewy et al. (1998) The human phase response curve (PRC) to melatonin is about 12 hours out of phase with the PRC to light. Chronobiol. Int. 15: 71-83.
Lewy et al. (1999) The endogenous melatonin profile as a marker for circadian phase position. J. Biol. Rhythms. 14: 227-236.
Lewy (1999) Melatonin as a marker and phase-resetter of circadian rhythms in humans. Adv. Exp. Med. Biol. 460: 425-434.
Masubuchi et al. (2000) Clock genes outside the suprachiasmatic nucleus involved in manifestation of locomotor activity rhythm in rats. Eur. J. Neurosci. 12: 4206-4214.
Mayer & Walker (eds.) (1987) Immunochemical Methods In Cell And Molecular Biology, Academic Press, London.
Mills et al. (1978a) Exogenous and endogenous influences on rhythms after sudden time shift. Ergonomics. 21: 755-761.
Mills et al. (1978b) Adaptation to abrupt time shifts of the oscillator(s) controlling human circadian rhythms. J. Physiol. 285: 455-470.
Miller & Calos (eds.) (1987) Gene Transfer Vectors For Mammalian Cells, Cold Spring Harbor Laboratory.
Minors et al. (1991) A human phase-response curve to light. Neurosci. Lett. 133: 36-40.
Minors & Waterhouse (1994) Deriving a “phase response curve” from adjustment to simulated time zone transitions. J. Biol. Rhythms. 9: 275-82.
Minors et al. (1994) The adjustment of the circadian rhythm of body temperature to simulated time-zone transitions: a comparison of the effect of using raw versus unmasked data. Chronobiol. Int. 11: 356-66.
Moore-Ede et al. (1982) The clocks that time us. Cambridge: Harvard University Press, 1982.
Perbal, (1984) A Practical Guide To Molecular Cloning.
Pittendrigh, Circadian rhythms and the circadian organization of living systems. In: Cold Spring Harbor Symposia on Quantitative Biology. Volume 25. Biological Clocks. pp. 159-184.
Pittendrigh & Daan (1976) A functional analysis of circadian pacemakers in nocturnal rodents. I. The stability and lability of spontanerous frequency. J. Comp. Phsyil. 106: 291-331.
Puchalski & Lynch (1991) Circadian characteristics of Djungarian hamsters: effects of photoperiodic pretreatment and artificial selection. Am. J. Physiol. 261: R670-676.
Remington (1995) The Science and Practice of Pharmacy, Mack Publishing Co., Easton, Pa.
Rajaratnam & Arendt (2001) Health in a 24-h society. Lancet. 358: 999-1005.
Robinson et al. (1991) The Feasibility of Repeated Microdialysis for With-In Subjects Design Experiments: Studies on the Mesostriatal Dopamine System. In: Microdialysis in the Neurosciences. Robinson, T. E., Justice, J. B., eds., Elsevier, Amsterdam, NY 189-234.
Roseboom et al. (1996) Melatonin synthesis: analysis of the more than 150-fold noctum increase in serotonin N-acetyltransferase mesenger ribonucleic acid in the rat pineal gland, Endocrinology, 137: 3033-3045.
Sambrook et al. (1989) Molecular Cloning: A Laboratory Manual, Second Edition Cold Spring Harbor Laboratory Press, Cold Spring Harbor, N.Y.
Spitzer et al. (1999) Jet lag: clinical features, validation of a new syndrome-specific scale, and lack of response to melatonin in a randomized, double-blind trial. Am. J. Psychiatry. 156: 1392-1396.
Summer et al. (1984) Phase-response and Aschoff illuminance curves for locomotor activity rhythm of the rat. Am. J. Physiol. 246: R299-304.
Sun et al. (2002) Circadian 5-HT production regulated by adrenergic signaling. Proc. Natl. Acad. Sci. USA. 99: 4686-4691.
Sun et al. (2003) Long-term in vivo pineal microdialysis. J. Pineal Res. 35: 118-24.
The Treatise, Methods In Enzymology, Academic Press, Inc., N.Y.
Ungerstedt, (1991) Introduction to Intracerebral Microdialysis. In: Microdialysis in the Neurosciences. Robinson, T. E., Justice, Jr., J. B., Eds. Elsevier, Amsterdam, NY 3-22.
Waterhouse et al. (1997) Jet-Lag. Lancet. 350: 1611-1616.
Weir & and Blackwell (eds.) (1986) Handbook Of Experimental Immunology, Volumes I-IV.
Wu et al. (eds.) Methods In Enzymology, Vols. 154 and 155.

1. A method for identifying at least one agent which modulates a preselected biological condition controlled by the circadian clock in a subject comprising
a) inserting a monitoring device into, or in close proximity to the pineal, causing little or no tissue damage to the non-pineal tissue during the inserting;
b) monitoring the chemical output of the pineal and monitoring a preselected biological condition of a first subject; and,
c) monitoring the chemical output of the pineal and monitoring the same preselected biological condition as in step b) in a second subject after contacting the second subject with the at least one agent;
wherein an alteration in the chemical output of the pineal and in the preselected biological condition in the second subject as compared to the chemical output of the pineal and preselected biological condition in the first subject identifies at least one agent which modulates a preselected biological condition controlled by the circadian clock.

2. The method of claim 1, wherein the monitoring of the chemical output is selected from the group consisting of in vivo microdialysis and ex vivo monitoring.

3. The method of claim 2, wherein the monitoring the chemical output comprises monitoring output of melatonin or serotonin (5-HT) or both.

4. The method of claim 1, wherein the preselected biological condition is subject behavior.

5. The method of claim 4, wherein the subject behavior is selected from the group consisting of symptoms of adaptation to new time zones, symptoms resulting from jet lag, symptoms of frequent shift work sleep abnormalities and symptoms of seasonal affective illnesses.

6. The method of claim 5, wherein the symptom is selected from the group consisting of a change in hormone secretion, a change in melatonin output, a change in sleep patterns, a change in activity patterns, a change in cortisol secretion and a change in core body temperature.

7. The method of claim 1, wherein the preselected biological condition is cellular expression of at least one biological molecule of interest.

8. The method of claim 1, wherein the preselected biological condition is tissue physiology.

9. The method of claim 1, wherein the monitoring is continuous, periodic, short term, long term, or any combination thereof.

10. The method of claim 1, wherein the monitoring is of a length of time sufficient to monitor one or more circadian rhythms of the subject.

11. The method of claim 1, wherein the first subject and the second subject are the same individual.

12. A composition comprising one or more agents and derivatives thereof identified by the method of claim 1.

13. An agent or derivative thereof identified by the method of claim 1 in purified form.

14. A pharmaceutically acceptable composition comprising one or more agents, or derivatives thereof, identified by the method of claim 1.

15. An improved method of carrying out surgery on the pineal comprising opening the skull of a subject and inserting a monitoring device, the improvement comprising a circular dental disk drill to open the skull, and a hook to lift and/or separate nonpineal tissues away from the pineal to allow visual placement of the monitoring device into, or in close proximity to, the pineal, causing little or no tissue damage to the non-pineal tissue during the inserting.

16. The method of claim 15, wherein the monitoring device is a microdialysis probe.

17. A method for implantation of a microdialysis probe for monitoring of chemicals produced by the pineal, comprising opening the skull and separating nonpineal tissue away from the pineal so as to visually expose the pineal, implanting a microdialysis probe into, or in close proximity to, the pineal, causing little or no tissue damage to the non-pineal tissue during the implanting.

18. A method for monitoring the presence of at least one chemical in the chemical output of the pineal comprising
a) opening the skull and visually exposing the pineal;
b) inserting a microdialysis probe into, or in close proximity to, the pineal, wherein non-pineal tissue exhibits little or no damage from the inserting;
c) contacting the pineal or the subject with at least one chemical; and,
d) monitoring the chemical output of the pineal for presence of the same or different chemical by in vivo microdialysis.

19. The method of claim 18, wherein the monitoring is long term, short term, continuous or periodic or any combination thereof.

20. A method of modulating a preselected condition controlled by the circadian clock in a subject in need thereof comprising
a) monitoring time of onset of melatonin secretion from said subject prior to presenting a light pulse to said subject;
b) presenting at least one light pulse to said subject, wherein said light pulse is presented during the subject's subjective night phase;
c) monitoring time of onset of melatonin secretion from said subject after said light pulse;
wherein when said melatonin secretion exhibits a shift in the time of onset of secretion after presentation of said light pulse, said preselected condition has been modulated.

21. The method of claim 20, wherein said light pulse is presented during the earlier half of the subjective night phase.

22. The method of claim 20, wherein said light pulse is presented during the later half of the subjective night phase.

23. The method of claim 20, wherein said preselected condition is selected from the group consisting of a change in hormone secretion, a change in melatonin output, a change in sleep patterns, a change in activity patterns, a change in cortisol secretion and a change in core body temperature.
